SEQUENCE LISTING (1 sequence)

SEQ ID NO: 1 - protein, 12 aa, synthetic construct
  REGION 1..12: note = SYNTHESIZED
GKPCNQFYCGGG

(19) United States
(12) Patent Application Publication
(10) Pub. No.: US 2024/0374674 A1
(43) Pub. Date: Nov. 14, 2024

(54) METHOD OF TREATING PATIENTS WITH HEPATORENAL SYNDROME TYPE 1

(71) Applicant: Mallinckrodt Pharmaceuticals Ireland Limited, Dublin (IE)

(72) Inventors: Khurram Jamil, Yardley, PA (US); Stephen Chris Pappas, The Woodlands, TX (US); Peter Teuber, Lebanon, NJ (US)

(21) Appl. No.: 18/783,556

(22) Filed: Jul. 25, 2024

Related U.S. Application Data
(62) Division of application No. 18/431,587, filed on Feb. 2, 2024.
Application No. 18/431,587 is a continuation of application No. 17/340,765, filed on Jun. 7, 2021.
Application No. 17/340,765 is a continuation-in-part of application No. 17/104,864, filed on Nov. 25, 2020.
Application No. 17/104,864 is a continuation-in-part of application No. 16/828,681, filed on Mar. 24, 2020, now abandoned.
Application No. 16/828,681 is a continuation-in-part of application No. 16/669,151, filed on Oct. 30, 2019, now abandoned.
Application No. 16/669,151 is a continuation-in-part of application No. 16/411,944, filed on May 14, 2019, now abandoned.
Application No. 16/411,944 is a division of application No. 14/920,392, filed on Oct. 22, 2015, now Pat. No. 10,335,452.
(60) Provisional application No. 62/151,384, filed on Apr. 22, 2015.
(60) Provisional application No. 62/068,357, filed on Oct. 24, 2014.

(51) Int. Cl. A61K 38/095; A61B 5/0205; A61B 5/145; A61K 9/00; A61K 38/38; A61K 45/06; A61M 27/00; G01N 15/01; G01N 15/10; G01N 33/49

(52) U.S. Cl. CPC A61K 38/095; A61B 5/0205; A61B 5/14542; A61K 9/0019; A61K 38/38; A61K 45/06; A61M 27/002; G01N 15/10; G01N 33/4925; G01N 2015/016; G01N 2015/1024

(57) ABSTRACT

The principles and embodiments of the present disclosure relate to methods for using terlipressin to treat a patient having impaired renal function associated with liver disease. A method of treating an adult patient with type 1 hepatorenal syndrome (HRS-1) may include administering a dose of terlipressin to the patient if the patient has a baseline model end stage liver disease (MELD) score <35, the patient has a serum creatinine (SCr) <5 mg/dl, the patient has an ACLF Grade <3, or a combination thereof and/or monitoring the patient's oxygenation level (SpO₂) before and during treatment with the terlipressin.

### CLAIM OF PRIORITY

This application is a divisional of U.S. patent application Ser. No. 18/431,587, filed on Feb. 2, 2024, which is a continuation of U.S. patent application Ser. No. 17/340,765, filed on Jun. 7, 2021, which is a continuation-in-part of U.S. patent application Ser. No. 17/104,864, filed on Nov. 25, 2020, which is a continuation-in-part of U.S. patent application Ser. No. 16/828,681, filed on Mar. 24, 2020, which is a continuation-in-part of U.S. patent application Ser. No. 16/669,151, filed on Oct. 30, 2019, which is a continuation-in-part of U.S. patent application Ser. No. 16/411,944, filed on May 14, 2019, which is a divisional application of U.S. patent application Ser. No. 14/920,392, filed on Oct. 22, 2015, which claims priority under 35 USC § 119 (e) to U.S. Patent Application Ser. No. 62/151,384, filed on Apr. 22, 2015, and U.S. Patent Application Ser. No. 62/068,357, filed on Oct. 24, 2014, the entire contents of which are hereby incorporated by reference.

### INCORPORATION OF SEQUENCE LISTING

The present application contains a Sequence Listing which has been submitted electronically in .XML format and is hereby incorporated herein by reference in its entirety. Said computer readable file, was created on Jan. 16, 2024 is named 105870_784351_Sequence_Listing.txt and is 4.096 kilobytes in size.

### TECHNICAL FIELD

Principles and embodiments of the present disclosure relate generally to methods of treating patients with type-1 hepatorenal syndrome.

### BACKGROUND

Hepatorenal Syndrome Type-1 (HRS Type 1 or HRS-1) is the development of acute kidney failure in patients with late-stage liver cirrhosis in the absence of any other cause. It is characterized by rapid onset of renal failure with a high mortality rate that exceeds 80% with within three months. Renal failure is an identified complication of cirrhosis of the liver; and, acute renal failure is known to have poor prognosis for patients with cirrhosis of the liver. In various instances, the renal failure may be caused by hypovolemia, hepatorenal syndrome without ongoing infection, or hepatorenal syndrome with an ongoing infection. Unfortunately, patients with HRS Type-1 may die from renal failure while waiting for a liver transplant. Currently, there is no way of determining which patients could maximally benefit from terlipressin treatment to reverse HRS Type-1.

Hepatorenal Syndrome (HRS) is indicated by low glomerular filtration rate due to renal vasoconstriction, splanchnic and peripheral arterial vasodilatation producing decreased vascular resistance, and portal hypertension. HRS is indicated by cirrhosis with ascites, serum levels of creatinine>133 μmol/l (1.5 mg/dL), no improvement of serum levels of creatinine (decrease to a level of ≤133 μmol/l) after at least 2 days of diuretic withdrawal and volume expansion with albumin, and the absence of shock and parenchymal kidney disease. HRS Type 1 is indicated by doubling of the initial serum levels of creatinine to >226 μmol/l (2.56 mg/dl) in <2 weeks.

Normal creatinine levels range from 0.7 to 1.3 mg/dl in men and 0.6 to 1.1 mg/dl in women. One mg/dl of creatinine equals 88.4 μmol/l.

Certain mechanisms that work to maintain effective arterial blood volume and relatively normal arterial pressure in patients with cirrhosis, however, affect kidney function, such as sodium and solute-free water retention, which can lead to ascites and edema, and to renal failure by causing intrarenal vasoconstriction and hypoperfusion. Ascites can result from the combination of portal hypertension and splanchnic arterial vasodilation that alters intestinal capillary pressure and permeability, which facilitates the accumulation of the retained fluid in the abdominal cavity.

A factor contributing to ascites formation is a splanchnic vasodilation that results in a decreased effective arterial blood volume. Portal hypertension also results from increased hepatic resistance to portal blood flow in cirrhotic livers, and may induce splanchnic vasodilation. There may be a marked impairment in solute-free renal water excretion and renal vasoconstriction, which leads to HRS.

In various instances, there may be signs of hepatic decompensation including INR>1.5, ascites, and encephalopathy. Hyponatremia is also a frequent complication of patients with cirrhosis and ascites that is associated with increased morbidity.

Systemic Inflammatory Response Syndrome (SIRS) is an inflammatory response that is not necessarily related to an infection, but may be due to nonspecific insults that initially produces local cytokines. SIRS is typically characterized by four criteria, including (1) core body temperature of less than 36° C. (96.8° F.) or greater than 38° C. (100.4° F.), (2) a heart rate of greater than 90 beats per minute, (3) tachypnea (high respiratory rate), with greater than 20 breaths per minute; or, an arterial partial pressure of carbon dioxide (CO₂) of less than 4.3 kPa (32 mmHg), and (4) a white blood cell count less than 4000 cells/mm³ (4×10⁹ cells/L) or greater than 12,000 cells/mm³ (12×10⁹ cells/L); or the presence of greater than 10% immature neutrophils (band forms) band forms greater than 3% is called bandemia or a “left-shift.” SIRS can be diagnosed when two or more of these criteria are present.

Sepsis has been defined as a systemic inflammatory response to infection, and septic shock is sepsis complicated by either hypotension that is refractory to fluid resuscitation or by hyperlactatemia.

The mortality of patients suffering from HRS and SIRS can be quite high, approaching 70%.

A number of studies have been conducted on patients having end-stage liver disease and systemic inflammatory responses. One such study described by Thabut et al., disclosed in HEPATOLOGY, Vol. 46, No. 6, 2007 entitled “Model for End-Stage Liver Disease Score and Systemic Inflammatory Response Are Major Prognostic Factors in Patients with Cirrhosis and Acute Functional Renal Failure”, which is incorporated herein by reference in its entirety, concluded that the presence of SIRS criteria with or without infection was a major independent prognostic factor in patients with cirrhosis and acute functional renal failure.

The presence of HRS and SIRS typically indicates a short life span if not effectively treated with the proper medication within a short span of time. It is therefore paramount that the most effective treatments for patients presenting with particular symptoms be identified and the patients started on an appropriate regimen as quickly as possible.

Terlipressin is a synthetic analogue of vasopressin having a prolonged effect, which acts as a peptidic vasopressin Vla receptor agonist. Terlipressin is a derivative of vasotocin prepared by extending the N-terminal by three amino acid residues, and used as a vasoactive drug in the management of hypotension. Terlipressin may be synthesized by coupling amino acids stepwise to one another in a liquid or solid phase with a peptide synthesizer. Terlipressin is a prodrug that slowly metabolizes to lysine-vasopressin and in this way provides prolonged biological effect. The half-life of terlipressin is 6 hours (the duration of action is 2-10 hr), as opposed to the short half-life of vasopressin, which is only 6 minutes (the duration of action is 30-60 min).

The chemical structure for terlipressin (Gly-Lys-Pro-Cys-Asn-Gln-Phe-Tyr-Cys-Gly-Gly-Gly; SEQ ID NO: 1) in an injectable formulation is show below.

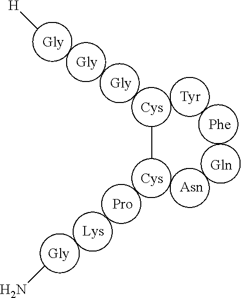

Molecular Formula: C₅₂H₇₄N₁₆O₁₅S₂

Molecular Weight: 1227.4 daltons

Appearance: Homogenous lyophilized white to off-white solid

Solubility: Clear, colorless solution in saline

Vials: Colorless glass vials containing 11 mg of a white to off-white solid, 1 mg active ingredient and 10 mg mannitol.

The active ingredient, N—[N—(N-glycylglycyl)glycyl]-8-L-lysinevasopressin, is a synthetically manufactured hormonogen of 8-lysine vasopressin, composed of 12 amino acids and having the characteristic ring structure of a cyclic nonapeptide with a disulfide bridge between the fourth and the ninth amino acid. Three glycyl-amino acids are substituted at position 1 (cysteine) of 8-lysine-vasopressin. By this N-terminal extension of 8-lysine-vasopressin the metabolic degradation rate of the active ingredient is significantly reduced, because the glycyl molecules inhibit rapid N-terminal enzymatic degradation. Terlipressin may be present in pharmaceutical compositions as a salt, diacetate salt, hydrate, and/or free base, such as terlipressin acetate or terlipressin diacetate pentahydrate.

### SUMMARY

Principles and embodiments of the present disclosure relate generally to methods of treating patients having HRS-1 by administering terlipressin to the patients to obtain reversal of the HRS-1. In one or more embodiments, response criteria provide a new and useful function of indicating a likelihood of improved response by a patient to the administration of terlipressin.

Some aspects of the disclosure relate to a method of treating a patient with hepatorenal syndrome Type 1 (HRS-1) comprising: administering a dose of terlipressin to the patient by intravenous (IV) injection, when the patient is listed for transplant at baseline and has a baseline model end stage liver disease (MELD) score of less than 35; and discontinuing administration or reducing the dose of terlipressin in patients with serum creatinine (SCr) ≥5 mg/dl and/or an acute-on-chronic liver failure (ACLF) Grade ≥3. The terlipressin may be administered every 6 hours by IV bolus injection over 2 minutes.

In some aspects, the method may further comprise acquiring the baseline MELD score of the patient. In additional aspects, the method may further comprising acquiring the SCr level in the patient prior to administering the dose of terlipressin to determine a baseline SCr level. In some examples, the terlipressin may not be administered if the baseline SCr >5 mg/dl and/or the baseline ACLF grade ≥3. In other aspects, the patient's risk of mortality is decreased. The patient's place on a transplant list may not be compromised or impacted by the administration of terlipressin. In some aspects, the patient may have an increased risk of their place on a transplant list being compromised or impacted due to the administration of terlipressin. In addition, the patient's ICU stay, non-ICU stay, and/or total length of hospital stay may be shortened. In some aspects, terlipressin administration is continued until there is a complete response or a partial response. In other aspects, discontinuing administration or reducing the dose of terlipressin occurs in patients with respiratory failure. The patient may also have severe kidney disease, pulmonary edema, dyspnea, or a combination thereof. In various aspects, the method may further comprise monitoring the patient's oxygen saturation during treatment with terlipressin. The monitoring of the oxygen saturation decreases the occurrence of adverse events.

Additional aspects of the disclosure relate to a method of treating a patient with hepatorenal syndrome Type 1 (HRS-1) comprising: administering a dose of terlipressin to the patient by intravenous (IV) injection when the patient has a baseline model end stage liver disease (MELD) score of less than 35 and is listed for transplant. The method may further comprise acquiring the baseline MELD score of the patient. In some aspects, the method may further comprise not administering, discontinuing administration, or reducing the dose of terlipressin in patients with serum creatinine (SCr) ≥5 mg/dl and/or an acute-on-chronic liver failure (ACLF) Grade ≥3.

Further aspects of the disclosure relate to a method of treating a patient with hepatorenal syndrome Type 1 (HRS-1) comprising: acquiring a baseline model end stage liver disease (MELD) score of the patient; acquiring a SCr level in the patient prior to administering a dose of terlipressin to determine a baseline SCr level; and administering a dose of terlipressin to the patient if the baseline MELD score is <35, the baseline SCr level is <5 mg/dl, the patient has an ACLF Grade <3, or a combination thereof. In some aspects, the patient may be listed for transplant. In additional aspects, the patient may have a MELD score >35 if the patient is not listed for transplant at baseline. The terlipressin may be administered to the patient by intravenous (IV) injection.

Yet further aspects of the disclosure relate to a method of treating a patient with hepatorenal syndrome Type 1 (HRS-1) comprising: administering a dose of terlipressin to a patient by intravenous (IV) injection, wherein administration only occurs if the patient is listed for transplant at baseline and has a baseline model end stage liver disease (MELD) score <35, the patient has a serum creatinine (SCr) <5 mg/dl, the patient has an ACLF Grade <3, or a combination thereof. In some aspects, the method may further comprise: monitoring the patient for fluid overload during treatment with the terlipressin; and reducing or discontinuing the terlipressin dose if fluid overload develops. In addition, the method may further comprise administering diuretics to the patient and/or measuring the SCr level in the patient.

Other aspects of the disclosure relate to a method of increasing overall survival of a patient with hepatorenal syndrome Type 1 (HRS-1), the method comprising: acquiring a baseline model end stage liver disease (MELD) score of the patient; acquiring a SCr level in the patient to determine a baseline SCr level; and administering a dose of terlipressin to the patient if the patient's baseline MELD score is <35 and the baseline SCr level is <5 mg/dl. In some aspects, the patient may be listed for transplant at baseline. In additional aspects, the patient may have a MELD score ≥35 if the patient is not listed for transplant at baseline.

Additional aspects of the disclosure relate to a method of decreasing an overall ICU or hospital stay of a patient with hepatorenal syndrome Type 1 (HRS-1), the method comprising: acquiring a baseline model end stage liver disease (MELD) score of the patient; acquiring a SCr level in the patient to determine a baseline SCr level; and administering a dose of terlipressin to the patient if the patient's baseline MELD score is <35 and the baseline SCr level is <5 mg/dl. In some aspects, the patient may be listed for transplant at baseline. In additional aspects, the patient may have a MELD score >35 if the patient is not listed for transplant at baseline.

Additional aspects of the disclosure relate to a method of increasing a complete response of a patient with hepatorenal syndrome Type 1 (HRS-1), the method comprising: acquiring a baseline model end stage liver disease (MELD) score of the patient; acquiring a SCr level in the patient to determine a baseline SCr level; administering a dose of terlipressin to a patient by intravenous (IV) injection if the patient's baseline MELD score is <35 and the patient's baseline SCr level is <5 mg/dl; measuring the SCr level in the patient during administration of terlipressin; and continuing administration of terlipressin until the patient's SCr level is ≤1.5 mg/dl. In some aspects, the patient may be listed for transplant at baseline. In additional aspects, the patient may have a MELD score ≥35 if the patient is not listed for transplant at baseline.

Further aspects of the disclosure relate to a method of increasing a partial response of a patient with hepatorenal syndrome Type 1 (HRS-1), the method comprising: acquiring a baseline model end stage liver disease (MELD) score of the patient; acquiring a SCr level in the patient to determine a baseline SCr level; administering a dose of terlipressin to a patient by intravenous (IV) injection if the patient's baseline MELD score is <35 and the patient's baseline SCr level is <5 mg/dl; measuring the SCr level in the patient during administration of terlipressin; and continuing administration of terlipressin until the patient experiences greater than 20% improvement in serum creatinine. The administration may be continued until the patient experiences greater than 30% improvement in serum creatinine. In some aspects, the patient may be listed for transplant at baseline. In additional aspects, the patient may have a MELD score ≥35 if the patient is not listed for transplant at baseline.

Other aspects of the disclosure relate to a method of treating a patient with hepatorenal syndrome Type 1 (HRS-1) comprising: administering a dose of terlipressin to the patient by intravenous (IV) injection, wherein the patient is excluded from treatment if the patient is listed for liver transplant with a MELD score ≥35.

Further aspects of the disclosure relate to a method of treating a patient with hepatorenal syndrome Type 1 (HRS-1) comprising: administering a dose of terlipressin to the patient by intravenous (IV) injection, wherein the patient is only treated if the patient belongs to a patient population that has median waiting time from listing to transplant of 5.6 months or greater.

Yet further aspects of the disclosure relate to a method of treating a patient with hepatorenal syndrome Type 1 (HRS-1) comprising: administering a dose of terlipressin to the patient by intravenous (IV) injection, wherein the patient is excluded from treatment if the patient belongs to a patient population that has median waiting time from listing to transplant of 0.23 months or less.

Some aspects of the disclosure relate to a method of treating a patient with hepatorenal syndrome Type 1 (HRS-1) comprising: narrowing the population of eligible patients for treatment to a mitigated population to reduce the risks selected from the group consisting of respiratory failure, serious adverse events, death, and combinations thereof; and administering a dose of terlipressin to the patient of the mitigated population by intravenous (IV) injection.

Additional aspects of the disclosure relate to a method of administering terlipressin to treat a patient with hepatorenal syndrome Type 1 (HRS-1), the patient being listed for transplant at a baseline model end state liver disease (MELD) score of less than 35, the method comprising: administering to the patient a dose of terlipressin by intravenous (IV) injection; and discontinuing administration or reducing the dose of terlipressin in patients with a serum creatinine (SCr) level ≥5 mg/dl and/or an acute-on-chronic liver failure (ACLF) Grade ≥3. In some aspects, the method may further comprise acquiring the SCr level in the patient prior to administering the dose of terlipressin to determine a baseline SCr level. The patient's risk of mortality may be decreased. The patient's place on a transplant list may not be compromised or impacted. The patient's ICU stay, non-ICU stay, and/or total length of hospital stay may be shortened. In some aspects, terlipressin administration is continued until there is a complete response or a partial response. In other aspects, discontinuing administration or reducing the dose of terlipressin occurs in patients with respiratory failure. The patient may also have severe kidney disease, pulmonary edema, dyspnea, or a combination thereof. The terlipressin may be administered every 6 hours by IV bolus injection over 2 minutes. In some aspects, the method may further comprise monitoring the patient's oxygenation level via pulse oximetry during treatment with the terlipressin. The monitoring of the oxygenation level decreases the occurrence of adverse events.

Some aspects of the disclosure relate to a method of treating a patient with hepatorenal syndrome Type 1 (HRS-1) comprising: obtaining a baseline oxygenation level (SpO₂) via pulse oximetry; administering a dose of terlipressin to the patient by intravenous (IV) injection if the patient is not experiencing hypoxia; and monitoring the patient's SpO₂ during treatment with the terlipressin. The monitoring of the oxygenation level may decrease the occurrence of adverse events. In some aspects, the patient's oxygen saturation is monitored for hypoxia. The method may further comprise discontinuing administration or reducing the dose of terlipressin if hypoxia is detected. For example, terlipressin may not be administered or may be discontinued if the patient's SpO₂ at baseline or during treatment is less than 90% and/or the patient's FiO₂ is greater than 0.36. The patient's SpO₂ may be monitored at least 3 times a day during administration of terlipressin.

Even further aspects of the disclosure relate to a method of treating a patient with hepatorenal syndrome Type 1 (HRS-1) comprising: administering a dose of terlipressin to a patient by intravenous (IV) injection; and discontinuing administration or reducing the dose of terlipressin in patients with serum creatinine (SCr) ≥5 mg/dl. Additional aspects of the disclosure relate to a method of treating a patient with hepatorenal syndrome Type 1 (HRS-1) comprising: measuring a serum creatinine (SCr) level in the patient; and administering a dose of terlipressin to the patient if the patient has serum creatinine (SCr) <5 mg/dl, the patient has an ACLF Grade <3, or a combination thereof. The terlipressin may be administered to the patient by intravenous (IV) injection. Further aspects of the disclosure include a method of treating a patient with hepatorenal syndrome Type 1 (HRS-1) comprising: administering a dose of terlipressin to a patient by intravenous (IV) injection, wherein administration does not occur if the patient has serum creatinine (SCr) >5 mg/dl, the patient has an ACLF Grade ≥3, or a combination thereof. In yet another aspect, the invention comprises a method of treating a patient with hepatorenal syndrome Type 1 (HRS-1) comprising: administering a dose of terlipressin to a patient by intravenous (IV) injection, wherein administration occurs only if the patient has serum creatinine (SCr) <5 mg/dl, the patient has an ACLF Grade <3, or a combination thereof. The method may further include monitoring the patient for fluid overload during treatment with terlipressin; and reducing the terlipressin treatment if fluid overload develops. As used herein, the terms “reducing the terlipressin treatment” and “reducing the terlipressin dose” may comprise lowering the terlipressin dose, interrupting the terlipressin dose, and/or not increasing the dose when the patient is previously prescribed or scheduled for an increased dose. Interrupting the terlipressin dose may include temporarily interrupting the dose until adverse events subside, until further notice, or until the patient has a serum creatinine (SCr) ≥5 mg/dl. In additional aspects, the method may further comprise measuring the SCr level in the patient. In some aspects, the patient's ICU stay, non-ICU stay, and/or total length of hospital stay may be shortened. The terlipressin administration may be continued until there is a complete response or a partial response.

In additional aspects, a method of treating a patient with hepatorenal syndrome Type 1 (HRS-1) comprises: administering a dose of terlipressin to a patient by intravenous (IV) injection; and managing fluid overload by reducing or discontinuing the administration of albumin, other fluids, and/or judicious use of diuretics. As used herein, judicious use of diuretics means the use of an effective amount of diuretics. For clarity, reducing the administration of albumin may comprise lowering the dose or interrupting the dose of albumin. If fluid overload persists, the method of treating may further comprise reducing or discontinuing terlipressin treatment. In a further aspect, managing fluid overload may decrease mortality or the occurrence of adverse events in the patient or patient population receiving treatment according to the invention described herein. In another aspect, the method of treating a patient with hepatorenal syndrome Type 1 (HRS-1) comprises: administering a dose of terlipressin to a patient by intravenous (IV) injection; and managing fluid overload by reducing or discontinuing the administration of albumin, other fluids, and/or judicious use of diuretics, wherein the patient additionally has respiratory failure, severe kidney disease, pulmonary edema, dyspnea, tachypnea, ischemia, or a combination thereof.

In additional aspects, a method of treating a patient with hepatorenal syndrome Type 1 (HRS-1) includes administering a dose of terlipressin to a patient by intravenous (IV) injection, wherein the dose of terlipressin is not increased in the presence of fluid overload, pneumonia, bronchospasm, pulmonary edema, ongoing significant adverse reactions, preexisting severe coronary artery disease, or combinations thereof. In additional aspects, a method of treating a patient with hepatorenal syndrome Type 1 (HRS-1) includes administering a dose of terlipressin to a patient by intravenous (IV) injection, wherein the dose of terlipressin is reduced or discontinued in the presence of fluid overload, pneumonia, bronchospasm, pulmonary edema, ongoing significant adverse reactions, preexisting severe coronary artery disease, or combinations thereof. Other aspects of the disclosure include a method of treating a patient with hepatorenal syndrome Type 1 (HRS-1) comprising: administering a dose of terlipressin to a patient every 6 hours by intravenous (IV) bolus injection over 2 minutes; monitoring the patient for fluid overload during treatment with terlipressin; and, reducing or discontinuing the terlipressin treatment if fluid overload persists.

Ischemic events (e.g., an inadequate blood supply to the skin, cardiac, vascular, or gastrointestinal tissues) may occur following administration of terlipressin. The most common ischemia-associated adverse events may comprise included skin discoloration, cyanosis, intestinal ischemia, and combinations thereof. Serious ischemic events in patients treated with terlipressin with may comprise intestinal ischemia, vascular skin disorder, cyanosis, livedo reticularis, myocardial infarction, poor peripheral circulation, myocardial ischemia, or combinations thereof. Terlipressin should be used with caution in patients with a history of ischemic events and certain cardiac conditions. In patients who experience signs or symptoms suggestive of ischemic adverse reactions, terlipressin dose should be reduced or permanently discontinued. Additional aspects of the disclosure relate to a method of treating a patient with hepatorenal syndrome Type 1 (HRS-1) comprising: administering a dose of terlipressin to a patient every 6 hours by intravenous (IV) bolus injection over 2 minutes, wherein the treatment of the patient with terlipressin is reduced or discontinued in the presence of ischemia. In another aspect, the invention comprises a method of treating a patient with hepatorenal syndrome Type 1 (HRS-1) comprising: administering a dose of terlipressin to a patient every 6 hours by intravenous (IV) bolus injection over 2 minutes, wherein the treatment of the patient with terlipressin is reduced or discontinued in the presence of skin, cardiac, vascular, or gastrointestinal ischemia, or combinations thereof in the patient. The terms “ischemia” and “ischemic events” may be used interchangeably.

Yet other aspects of the disclosure relate to a method of treating a patient with hepatorenal syndrome Type 1 (HRS-1) comprising: administering a dose of terlipressin to a patient every 6 hours by intravenous (IV) bolus injection over 2 minutes, wherein the dose of terlipressin is not increased in the presence of fluid overload, pneumonia, bronchospasm, or pulmonary edema. Further aspects of the disclosure relate to a method of treating a patient with hepatorenal syndrome Type 1 (HRS-1) comprising: administering a dose of terlipressin to a patient every 6 hours by intravenous (IV) bolus injection over 2 minutes; monitoring the patient for fluid overload during treatment with terlipressin; and, reducing or discontinuing the dose of terlipressin if fluid overload develops. In yet another aspect of the disclosure, a method of treating a patient with hepatorenal syndrome Type 1 (HRS-1) includes: administering a dose of terlipressin to a patient every 6 hours by intravenous (IV) bolus injection over 2 minutes, wherein the treatment with terlipressin is immediately interrupted in the presence of treatment-emergent pulmonary edema, new onset or worsening pneumonia, or unresolved hepatic encephalopathy ≥Grade 3 with risk of aspiration.

Further aspects of the disclosure relate to a method of increasing overall survival or a patient with hepatorenal syndrome Type 1 (HRS-1), the method comprising: measuring a serum creatinine (SCr) level in the patient; and administering a dose of terlipressin to the patient if the patient has serum creatinine (SCr) <5 mg/dl. More aspects of the disclosure relate to a method of decreasing an overall ICU or hospital stay of a patient with hepatorenal syndrome Type 1 (HRS-1), the method comprising: measuring a serum creatinine (SCr) level in the patient; and administering a dose of terlipressin to the patient if the patient has SCr <5 mg/dl.

Additional aspects of the disclosure relate to a method of increasing a complete response of a patient with hepatorenal syndrome Type 1 (HRS-1), the method comprising: measuring a serum creatinine (SCr) level in the patient; administering a dose of terlipressin to a patient by intravenous (IV) injection if the patient's SCr level is <5 mg/dl; and continuing administration of terlipressin until the patient's SCr level is ≤1.5 mg/dl. Yet further aspects of the disclosure relate to a method of increasing a partial response of a patient with hepatorenal syndrome Type 1 (HRS-1), the method comprising: measuring a serum creatinine (SCr) level in the patient; administering a dose of terlipressin to a patient by intravenous (IV) injection if the patient's SCr level is <5 mg/dl; and continuing administration of terlipressin until the patient experiences greater than 20% improvement in serum creatinine.

Additional aspects and features are set forth in part in the description that follows, and will become apparent to those skilled in the art upon examination of the specification or may be learned by the practice of the disclosed subject matter.

### BRIEF DESCRIPTION OF THE DRAWINGS

Further features of embodiment of the present disclosure, their nature and various advantages will become more apparent upon consideration of the following detailed description, taken in conjunction with the accompanying drawings, which are also illustrative of the best mode contemplated by the applicants, and in which like reference characters refer to like parts throughout, where:

FIG. 1 illustrates an exemplary embodiment of a terlipressin treatment protocol;

FIG. 2 illustrates an exemplary embodiment of a terlipressin treatment protocol;

FIG. 3 illustrates a set of unexpected results from an exemplary embodiment of a terlipressin treatment protocol;

FIG. 4 illustrates an exemplary embodiment of a terlipressin treatment protocol;

FIG. 5 is a graph showing 90-day survival of patients treated with and without terlipressin according to ACLF grade;

FIG. 6A shows the percentage of patients with renal failure at baseline and end of treatment for patients treated with terlipressin and placebo;

FIG. 6B shows the percentage of patients with respiratory failure at baseline and end of treatment for patients treated with terlipressin and placebo; and

FIG. 6C shows the percentage of patients with respiratory failure with ACLF Grade ≤2 and ACLF Grade ≥3 for patients treated with terlipressin and placebo.

### DETAILED DESCRIPTION

The principles and embodiments of the present disclosure relate to methods of improving a patient's renal condition involving a treatment protocol comprising terlipressin. Accordingly, various embodiments of the present disclosure provide methods of treating a patient with terlipressin or terlipressin and albumin.

As used herein, use of “terlipressin” may refer to terlipressin or salts, diacetate salts, hydrates, and/or free bases thereof. For example, use of terlipressin may include terlipressin acetate or terlipressin diacetate pentahydrate. In additional examples, terlipressin may refer to any other suitable salts or hydrates thereof or any other biologically acceptable salts or hydrates thereof.

As used herein, the terms “reducing the terlipressin treatment” and “reducing the terlipressin dose” may comprise lowering the terlipressin dose, interrupting the terlipressin dose, and/or not increasing the dose when the patient is previously prescribed or scheduled for an increased dose. Interrupting the terlipressin dose may include temporarily interrupting the dose until adverse events subside, until further notice, or until the patient has a serum creatinine (SCr) ≥5 mg/dl.

As used herein, the “mitigated population” or “subset of patients” includes HRS-1 patients with a baseline ACLF Grade 0-2, a baseline serum creatinine <5 mg/dL, and/or a baseline MELD <35. For example, the mitigated population excludes patients with baseline ACLF Grade 3, baseline serum creatinine ≥5 mg/dl, and/or patients listed for transplant at baseline with a baseline MELD ≥35.

In embodiments of the present disclosure, the patient is evaluated to determine the particular disease and/or syndrome he or she may be suffering from, and beginning a treatment regimen for patients that will benefit from the administration of terlipressin.

In various embodiments, the patient has end stage liver disease complicated with acute kidney failure, such as HRS, and is treated with terlipressin.

In various embodiments, end-stage liver disease may be cirrhosis of the liver or fulminant liver failure. In various embodiments, the end-stage liver disease is complicated by impaired renal function.

HRS-1 in decompensated cirrhosis is related to hemodynamic abnormalities. Terlipressin improves renal perfusion in HRS-1 by enhancing intravascular volume through splanchnic vasoconstriction. In some aspects, terlipressin may be more effective than placebo in albumin-treated patients with decompensated cirrhosis and HRS-1. An aspect of the present disclosure relates to a method of diagnosis of patients that show improved response to terlipressin treatment, as indicated by an increased probability of HRS reversal.

In one or more embodiments, the method of identifying an HRS-1 patient with an increased likelihood of responding to terlipressin treatment regimen comprises identifying a patient as having end stage live disease and impaired renal function, determining if the patient also exhibits at least two out of three criteria for SIRS, wherein the three response criteria include (1) a white blood cell count (WBC) that is less than 4,000 cells/mm³ or greater than 12,000 cells/mm³, (2) a heart rate of greater than 90 beats per minute (BPM), and (3) an HCO₃<21 mmol/L, where HCO₃ is considered a surrogate measurement that approximates the response criteria of arterial partial pressure of carbon dioxide (PaCO₂)<32 mmHg. In various embodiments, a heart rate of >85 BPM and/or an HCO₃<23 mmol/L may be applied as the response criteria.

An aspect of the present disclosure relates to terlipressin for use in the treatment of HRS-1 in a subject that is also exhibiting at least two of the following three response criteria:
(a) a white blood cell count (WBC) is less than 4,000 cells/mm³ or greater than 12,000 cells/mm³,
(b) a heart rate of greater than 90 beats per minute (BPM), and
(c) an HCO₃<21 mmol/L, where HCOs is considered a surrogate measurement that approximates the response criteria of arterial partial pressure of carbon dioxide (PaCO₂)<32 mmHg. In various embodiments, one or more single dosages of terlipressin is administered to the subject, thereby treating the HRS-1.

In various embodiments, the terlipressin dosage is administered to the patient in the range of about 0.5 mg to about 2.0 mg every 4 to 6 hours, as a series of single doses, so that the patient receives a single dose in the range of about 0.5 mg to about 2.0 mg of terlipressin followed by another single dose 4 to 6 hours later. In various embodiments, a patient may receive 4 to 6 doses over a 24 hour period, where each dose is in the range of about 0.5 mg to about 2.0 mg. In various embodiments, the total dosage does not exceed 4.0 mg over a 24 hour period.

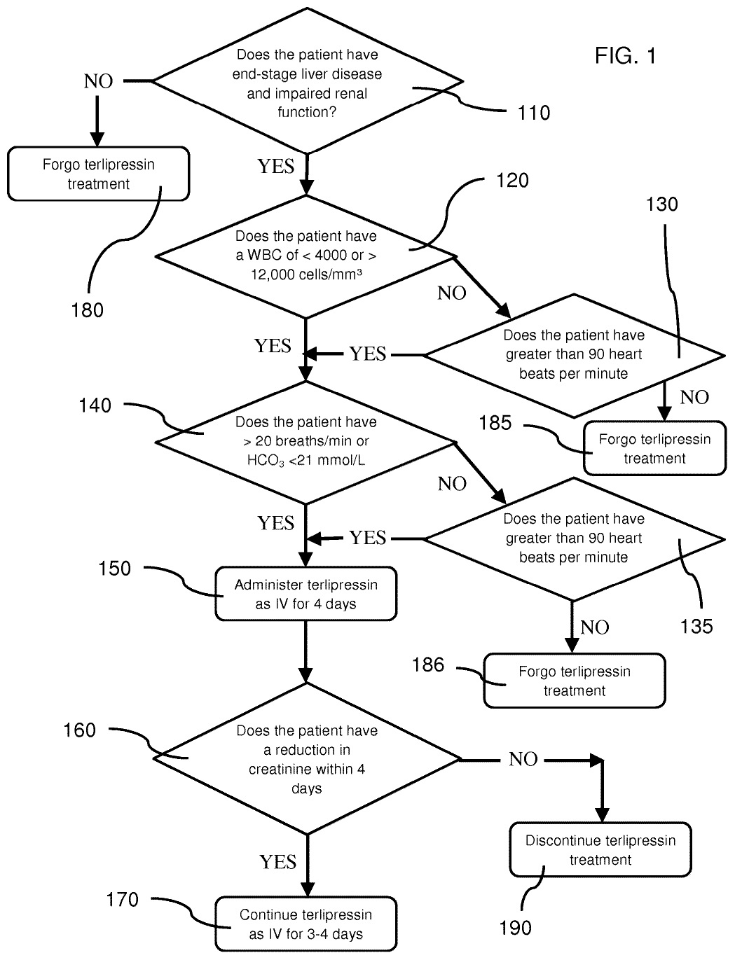

As shown in FIG. 1, an exemplary embodiment of a method of treating a patient via an embodiment of a terlipressin treatment protocol.

In various embodiments, a patient, who is initially identified as having end stage liver disease, for which treatment with a vasodilator may provide an improvement in renal function, is tested to determine the extent of the patient's cirrhosis and renal failure.

At 110, a patient is initially identified as having end stage liver disease and impaired renal function. In various embodiments, a patient may be suffering from cirrhosis of the liver or fulminant liver failure, where a patient identified with cirrhosis of the liver may have a Child-Pugh score of A, B, or C. In various embodiments, a patient identified with cirrhosis of the liver that has a Child-Pugh score of B or C may be considered a viable candidate for terlipressin treatment. In various embodiments, a patient identified with cirrhosis of the liver that has a Child-Pugh score of C may be considered a viable candidate for terlipressin treatment. Various complications of end-stage liver disease, and in particular cirrhosis, are recognized and have a notably poor prognosis.

In one or more embodiments, a treatment protocol comprising dosages of terlipressin surprisingly provides reversal of one or more complicating factors, such as vasodilation, and reduces mortality from the associated complications within a 90 day window starting with treatment.

In one or more embodiments, the terlipressin treatment protocol comprises identifying a patient having end-stage liver disease and impaired renal function, where the identified patient may benefit from a treatment comprising administration of terlipressin, determining if the patient also exhibits at least two out of three response criteria, excluding the patient from administration of terlipressin if the patient exhibits uncontrolled infection, sepsis, or septic shock is excluded from the terlipressin treatment, and initiating terlipressin treatment by administering a daily dosage of terlipressin to the patient in an amount effective to produce an improvement in renal function, wherein an improvement in renal function is indicated by a reduction in SCr of at least 25% from baseline, reversal of HRS (defined as a decrease in SCr level to ≤1.5 mg/dl), and/or confirmed HRS reversal (defined as two serum creatinine values of ≤1.5 mg/dL at least 48 hours apart)).

In one or more embodiments, the patient is alive at day 90 after initiating terlipressin treatment. For example, a patient that experiences HRS reversal, verified HRS reversal, and/or greater than 30% improvement in SCr after receiving terlipressin may have at least a 60%, 65%, or 70% likelihood of being alive at day 90. In other embodiments, the patient is alive and transplant-free at day 90 after initiating terlipressin treatment. For example, a patient that experiences HRS reversal, verified HRS reversal, and/or greater than 30% improvement in SCr after receiving terlipressin may have at least a 35%, 40%, or 45% likelihood of being alive and transplant-free at day 90.

In one or more embodiments, the terlipressin dosage may be in the range of about mg to about 10 mg, or 0.5 mg to about 5.0 mg, or 0.5 mg to about 2.0 mg, or 0.5 mg to about mg, or about 1.0 mg to about 2.0 mg per single administration. In various embodiments, the injections may be administered intravenously as slow bolus injections over 2 minutes, where the dose may be repeated every four to six hours. If on day 4 of therapy (after a minimum of 10 doses), SCr had decreased, but by less than 30% from the baseline value, the dose may be increased to 2 mg every 6 hours (+30 min) (8 mg/day). The dose may not be increased if the subject had coronary artery disease; or in the clinical setting of circulatory overload, pulmonary edema, or treatment-refractory bronchospasm. The terms “circulatory overload” and “fluid overload” may be used interchangeably. In various embodiments, if dosing was interrupted due to a non-ischemic adverse event, terlipressin may be restarted at the same or lower dose (i.e., 0.5 to 1 mg q6h).

At 180, a patient that is not diagnosed with an end-stage liver disease and impairment of renal function is excluded from the terlipressin treatment.

In one or more embodiments, the patient is tested for three specific response criteria, where the criteria include a determination of (1) whether the white blood cell count (WBC) is less than 4,000 cells/mm³ or greater than 12,000 cells/mm³, (2) whether the patient has a heart rate of greater than 90 beats per minute (BPM), and/or (3) whether the patient has tachypnea with greater than 20 breaths per minute or an HCO₃<21 mmol/L, where HCO₃ is considered a surrogate measurement that approximates the response criteria of arterial partial pressure of carbon dioxide (PaCO₂)<32 mmHg. In various embodiments, the response criterion of a patient's core body temperature being less than 36° C. (96.8° F.) or greater than 38° C. (100.4° F.) is not measured or considered in determining if the patient has two or more response criteria. In some examples, the response criteria may be SIRS criteria. In various embodiments, the criteria may be tested in any order.

At 120, a patient is tested to determine if the patient's WBC is <4,000 or >12,000 cells/mm³. In various embodiments, the testing is specifically directed at determining if the patient's leukocytes are less than 4000 cells/mm³ (4×10⁹ cells/L) or greater than 12,000 cells/mm³ (12×10⁹ cells/L). In various embodiments, a patient will be considered to meet the response criterion if the patient's WBC is <5,000 or >12,000 cells/mm³. In various embodiments, the patient is not tested for the presence of greater than 10% immature neutrophils (band forms). In various embodiments, the testing method to determine the WBC may be any of the methods known in the art.

If the patient is found to not have a WBC outside the range of 4,000 to 12,000 cells/mm³, the patient may still be diagnosed with SIRS if the patient meets the two other response criteria.

In various embodiments, a patient that has a WBC<4,000 or >12,000 cells/mm³ is considered to meet that response criterion.

At 130, a patient that does not have a WBC outside the range of 4,000 to 12,000 cells/mm³ is tested to determine if the patient's heart rate is >90 BPM. If the patient's heart rate is >90 BPM, the patient will be considered to meet that response criterion. In various embodiments, a patient with a heart rate of >85 BPM will be considered to meet that response criterion. The testing method to determine the patient's heart rate may be any of the methods known in the art.

In various embodiments, a patient that has a WBC outside the range of 5,000 to 12,000 cells/mm³ is tested to determine if the patient's heart rate is >90 BPM. If the patient's heart rate is >90 BPM, the patient will be considered to meet that response criterion. In various embodiments, a patient with a heart rate of >85 BPM will be considered to meet that response criterion.

At 185, a patient that does not exhibit both a WBC<4,000 or >12,000 cells/mm³ and a heart rate that is >90 BPM is considered to not qualify for two of the three response criteria, and therefore does not meet the requirements to be treated with terlipressin. A patient failing to meet at least two of the three response criteria is excluded from the terlipressin treatment. Such a patient may be treated instead with one or more other pharmacological agents such as nor-epinephrine, vasopressin, or a combination of midodrine and octreotide. Alternatively or in addition, any of the following may be used: N-acetylcysteine, misoprostol, and/or BQ123. Another option is transjugular intrahepatic portosystemic shunt (TIPS). Renal support in the form of dialysis is commonly instituted to manage acute fluid overload in HRS-1 patients, particularly if pharmacological therapies fail. The only effective and permanent treatment for end-stage cirrhosis and HRS is liver transplantation.

At 140, a patient that has a WBC outside the range of 4,000 to 12,000 cells/mm³ or a heart rate that is >90 BPM is tested to determine if the patient has >20 breaths per minute or an HCO₃<21 mmol/L. If the patient has >20 breaths per minute or an HCO₃<21 mmol/L, the patient will be considered to meet that response criterion. In various embodiments, a patient with an HCO₃<23 mmol/L will be considered to meet that response criterion. The testing method to determine the patient's breathing rate or HCOs may be any of the methods known in the art.

In various embodiments, a patient that has a WBC outside the range of 5,000 to 12,000 cells/mm³ is tested to determine if the patient has a breathing rate that is >20 breaths per minute or an HCO₃<21 mmol/L. If the patient has a breathing rate that is >20 breaths per minute or an HCO₃<21 mmol/L, the patient will be considered to meet that response criterion. In various embodiments, a patient with an HCO₃<23 mmol/L will be considered to meet that response criterion.

In one or more embodiments, if the patient has a WBC outside the range of 4,000 to 12,000 cells/mm³ and the patient has >20 breaths per minute or an HCO₃<21 mmol/L, the patient is considered to qualify for two of the three response criteria, and therefore meets the requirements to be treated with terlipressin unless otherwise excluded.

In one or more embodiments, if the patient has a heart rate that is >90 BPM and the patient has a breathing rate that is >20 breaths per minute or an HCO₃<21 mmol/L, the patient is considered to qualify for two of the three response criteria, and therefore meets the requirements to be treated with terlipressin unless otherwise excluded.

At 135, a patient that has a WBC outside the range of 4,000 to 12,000 cells/mm³, but does not have >20 breaths per minute or an HCO₃<21 mmol/L, is tested to determine if the patient's heart rate is >90 BPM. If the patient's heart rate is >90 BPM, the patient will be considered to meet that response criterion. In various embodiments, a patient with a heart rate of >85 BPM will be considered to meet that response criterion.

In one or more embodiments, in which the patient has a WBC outside the range of 5,000 to 12,000 cells/mm³, but the patient does not have >20 breaths per minute or an HCO₃<21 mmol/L, the patient is tested to determine if the patient's heart rate is >90 BPM. If the patient's heart rate is >90 BPM, the patient will be considered to meet that response criterion. In various embodiments, a patient with a heart rate of >85 BPM will be considered to meet that response criterion.

In one or more embodiments, if the patient has a breathing rate that is >20 breaths per minute or an HCO₃<21 mmol/L a heart rate that is >90 BPM and the patient has a breathing rate that is >20 breaths per minute or an HCO₃<21 mmol/L, the patient is considered to qualify for two of the three response criteria, and therefore meets the requirements to be treated with terlipressin unless otherwise excluded.

At 186, a patient that does not exhibit (1) a breathing rate that is >20 breaths per minute or an HCO₃<21 mmol/L and does not exhibit (2) a heart rate that is >90 BPM is considered to not qualify for at least two of the three response criteria, and therefore does not meet the requirements to be treated with terlipressin. A patient failing to meet at least two of the three response criteria is excluded from the terlipressin treatment. Optional alternative treatments for such a patient are described above.

While the tests for the response criteria have be discussed in a particular order for the exemplary embodiment, the tests may be done in any particular order.

In one or more embodiments, temperature is not a response criterion because patient temperature may not provide an accurate indication of patient response to terlipressin. In various embodiments, patient temperatures are excluded from the set of response criteria.

At 150, a patient that has end stage liver disease with impaired renal function, and qualifies for at least two of the three response criteria, is started on terlipressin. In one or more embodiments, a patient with uncontrolled infection, sepsis, or septic shock is excluded from the terlipressin treatment. In various embodiments, terlipressin is administered to the patient for one to four days. In various embodiments, the patient is administered terlipressin for four days unless the patient experiences an adverse event. In various embodiments, the terlipressin is administered to the patient as an IV drip.

In one or more embodiments, the terlipressin treatment protocol comprises administering a dosage of terlipressin in the range of about 0.1 mg to about 10 mg, or 0.5 mg to about 5.0 mg, or 0.5 mg to about 2.0 mg, or about 0.5 mg to about 1.0 mg, or about 1.0 mg to about 2.0 mg to the patient over about four hours to about six hours as an IV drip.

In one or more embodiments, the patient is administered terlipressin as an IV about every 4 to 6 hours for 1 to 4 days. In various embodiments, the terlipressin may be administered for at least 4 days.

In one or more embodiments, the patient is administered terlipressin as a slow bolus over 2 minutes about every 4 to 6 hours for 1 to 4 days. In various embodiments, the terlipressin may be administered for at least 4 days.

At 160, the patient that is being administered the terlipressin is tested at least once during the one to four day period of administration to determine if the patient is responding to the terlipressin. In various embodiments, the patient may be tested once prior to beginning the administration of the terlipressin to establish a baseline and once during the one to four days of terlipressin administration, or once prior to beginning the administration of the terlipressin to establish a baseline and once at the end of the four days of administration of the terlipressin. In various embodiments, the patient's creatinine levels are measured to determine if there has been a reduction in the patient's serum creatinine, where a reduction in serum creatinine levels of about 1.0 mg/dL or greater, or in the range of about 1.0 mg/dl to about 2.0 mg/dL, or a reduction of about 1.7 mg/dL from the patient's initial baseline value indicates an improvement in renal function and that the patient is responding to the terlipressin.

In various embodiments, improvement in renal function is indicted by a decrease in serum creatinine level of about 25% or about 30% in the patient receiving terlipressin.

In one or more embodiments, a patient may have his or her serum creatinine levels measured once a day or once every other day for each of the four day period after administration of terlipressin has begun, wherein a measurement made on the first day of terlipressin administration may be recorded and used as the baseline creatinine level.

In various embodiments, the method may comprises testing the patient's SCr level during the 1 to 4 days of terlipressin administration and determining if the patient has a reduction in SCr level by the end of the 1 to 4 days of terlipressin administration.

The serum creatinine levels may be measured by any of the methods known in the art, for example, the Jaffe reaction using alkaline picrate.

The GFR may be measured directly by clearance studies of exogenous markers, such as inulin, iohexol, iothalamate, and Cr51-EDTA, or by estimated glomerular filtration rate (eGFR) using creatinine testing methods that are traceable to a reference method based on isotope dilution-mass spectrometry (IDMS).

At 170, a patient that shows a positive response to the administration of the terlipressin evidenced by a reduction in the patient's serum creatinine level is continued on the terlipressin at the dosage in the range of about 0.1 mg to about 10 mg, or 0.5 mg to about 5.0 mg, or 0.5 mg to about 2.0 mg, or about 0.5 mg to about 1.0 mg, or about 1.0 mg to about 2.0 mg. In various embodiments, the dosage administered to the patient may be adjusted based upon the measured serum creatinine level(s). In various embodiments, a patient being administered terlipressin may have their serum creatinine levels monitored for the entire time period that the patient is receiving terlipressin. In one or more embodiments, the patient's serum creatinine level may be tested every day, or every other day, or every third day, or every fourth day, to confirm that the patient is still responding positively to the terlipressin treatment.

In various embodiments, the patient's terlipressin dosage may be increased from about 0.5 mg to about 1.0 mg to about 1.0 mg to about 2.0 mg after 2-3 days of terlipressin administration to the patient if there is <1.5 mg/dl decrease in SCr during the first 2-3 days of treatment.

In various embodiments, the dosage may be repeated every four to six hours for a time period of one or more days until the patient shows recovery, or until the patient no longer shows improvement. The terlipressin may be administered to the patient for a time period in the range of about two days to about sixteen days, or for a time period in the range of about four days to about eight days. In various embodiments, the time period is in the range of about seven days. In various embodiments, the terlipressin treatment may be continued until there is a complete response. In various embodiments, the duration of treatment of a patient with terlipressin may be 1 to 28 days.

At 190, a patient that does not show any improvement by the end of four days may have the terlipressin discontinued, where improvement is indicated by a decrease in serum creatinine levels over the one to four days the terlipressin is administered. In various embodiments the patient may be tested on the third or fourth day after starting treatment with the terlipressin to determine if there is a decrease in serum creatinine levels indicating a response to the treatment.

In one or more embodiments, a patient is provided 2 days of anti-infective therapy for documented or suspected infection before starting administration of terlipressin if an infection is suspected. In various embodiments, a patient may be started on the terlipressin treatment protocol after the patient has been administered the anti-infective therapy.

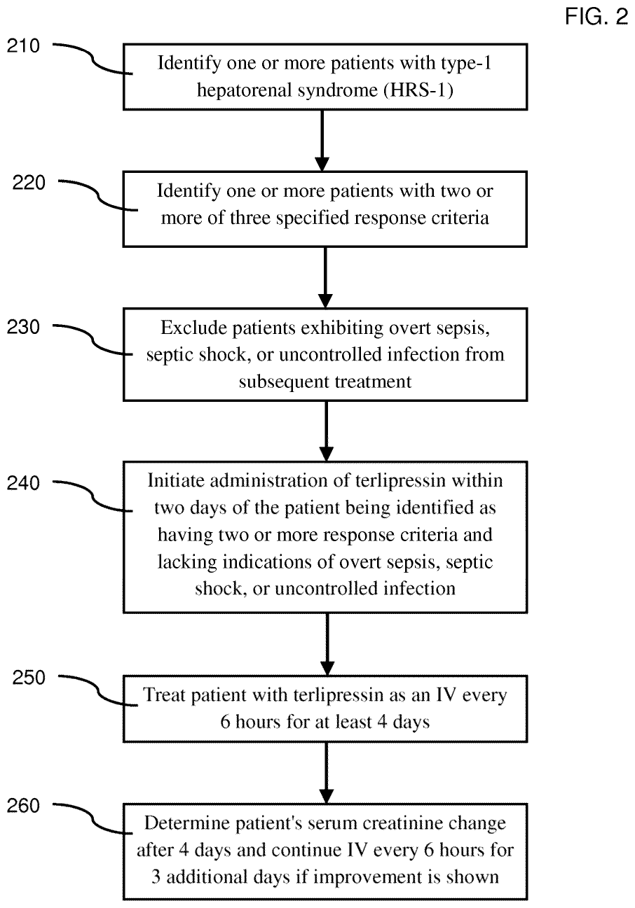

FIG. 2 illustrates an exemplary embodiment of a terlipressin treatment protocol.

Principles and embodiments of the present disclosure also relate to providing terlipressin as an IV every four to six hours to patients that have been identified with HRS-1 and two or more of three specific response criteria.

In one or more embodiments, a patient is tested for (1) a white blood cell count (WBC)<4 or >12 cells/μL; (2) a heart rate (HR)>90 beats per minute (bpm), and (3) HCO₃<21 mmol/L.

A non-SIRS patient is defined as subjects with <2 of the response criteria described above.

In various embodiments, temperature is not used as a response criterion.

In one or more embodiments of the disclosure, terlipressin is administered to patients presenting with a particular set of symptoms to mitigate the vasoconstriction in the kidneys, and improve renal function as indicated by a reduction in serum creatinine levels of about 1.7 mg/dL from initial baseline.

At 210, one or more patients that may be presenting with end-stage liver disease are tested to determine whether they are suffering from cirrhosis with ascites, and have serum levels of creatinine>133 μmol/l. A patient identified as having HRS is further tested and/or their medical history checked to determine if the initial serum levels of creatinine have doubled to greater than 226 μmol/l in less than 2 weeks indicating type 1 HRS.

Patients having HRS-1 and at least two of three response criteria have surprisingly shown improved response to terlipressin treatment compared to non-SIRS HRS-1 patients, as indicated by reversal of the HRS indications. In addition, patients having HRS-1, at least two of three response criteria, and not having uncontrolled infection, sepsis, or septic shock have surprisingly shown improved response to terlipressin treatment compared to non-SIRS HRS-1 patients. The HRS indications may include serum creatinine levels.

The patients having HRS-1 and SIRS may experience HRS reversal, verified HRS reversal, or greater than 30% improvement in SCr after receiving terlipressin. In one or more embodiments, the patent is alive at day 90 after initiating terlipressin treatment. For example, a patient that experiences HRS reversal, verified HRS reversal, and/or greater than 30% improvement in SCr after receiving terlipressin may have at least a 60%, 65%, or 70% likelihood of being alive at day 90. In other embodiments, the patient is alive and transplant-free at day 90 after initiating terlipressin treatment. For example, a patient that experiences HRS reversal, verified HRS reversal, and/or greater than 30% improvement in SCr after receiving terlipressin may have at least a 35%, 40%, or 45% likelihood of being alive and transplant-free at day 90.

At 220, once a patient has been identified as suffering from HRS-1, the patient is tested to determine is the same patient is exhibiting at least two out of three criteria indicating SIRS, wherein the three criteria include a (1) WBC<4 or >12 cells/μL; (2) HR>90 bpm, and (3) HCO₃<21 mmol/L.

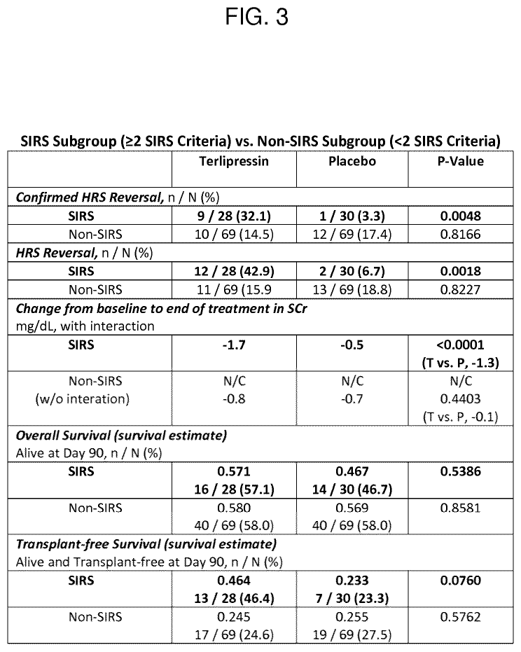

In various embodiments, patients not identified as exhibiting at least two of the three response criteria in addition HRS-1 are excluded from the terlipressin treatment protocol. Patients having HRS-1 and exhibiting at least two of the three response criteria have surprisingly shown improved response to terlipressin treatment compared to non-SIRS HRS-1 patients, as indicated by reversal of the HRS indications, as shown in FIG. 3.

At 230, patients that have been identified as having HRS-1 and exhibit at least two response criteria are tested to determine if they may also have an uncontrolled infection, sepsis, or septic shock, wherein patients identified as exhibiting an uncontrolled infection, sepsis, or septic shock are excluded from the terlipressin treatment protocol.

At 240, patients that have HRS-1, have at least two of the three response criteria, and do not have an uncontrolled infection, sepsis, or septic shock are started on the terlipressin treatment. In one or more embodiments, the terlipressin treatment is started within 48 hours of the initial diagnosis that the patient has both HRS-1 and at least two of three response criteria. In various embodiments, in which the determination that the patient does or does not also have an uncontrolled infection, sepsis, or septic shock occurs after 48 hours of the initial diagnosis of both HRS-1 and the response criteria, the treatment protocol is started within 48 hours of the initial diagnosis, and treatment may be terminated once an uncontrolled infection, sepsis, or septic shock manifests or is determined.

In various embodiments, a baseline serum creatinine level may be determined for the patient prior to starting the administration of terlipressin to the patient; and the administration of terlipressin started within 2 days or within 3 days, or within 4 days of determining the baseline serum creatinine level. In various embodiments, the patient may be tested at least once daily within four days after starting the administration of terlipressin to determine if the patient exhibits a decrease in the serum creatinine level compared to the previously determined baseline serum creatinine level.

At 250, terlipressin treatment of the patient is started and the patient receives a dosage of terlipressin. In one or more embodiments, the terlipressin may be administered to a patient as a slow infusion over 24 hours, wherein the dosage over the 24 hour period may be in the range of about 2.0 mg to about 12 mg. In various embodiments, the dosage over the 24 hour period may be in the range of about 2.0 mg to about 4.0 mg. In various embodiments, the terlipressin is administered as a continuous intravenous (IV) drip lasting from about 4 hours to about 6 hours, and comprising a dosage of about 0.5 mg to about 2.0 mg.

In one or more embodiments, the terlipressin dosage may be a dosage of about 0.5 mg to about 2.0 mg administered intravenously every 4 to 6 hours as a slow bolus injection over 2 minutes. The dosage may be administered about every 4 hours, about every 5 hours, about every 6 hours, about every 7 hours, about every 8 hours, about every 9 hours, about every 10 hours, about every 11 hours, or about every 12 hours by slow IV bolus injection. In at least one example, the dosage may be administered about every 6 hours by slow IV bolus injection. The bolus injection may be given over about 1 minute, about 2 minutes, about 3 minutes, about 4 minutes, or about 5 minutes. In at least one example, the bolus injection may be given over about 2 minutes.

In one or more embodiments, the terlipressin is used to treat the patient exhibiting HRS-1 and at least two of the three response criteria. In various embodiments, the patient is also tested to determine that the patient does not have an uncontrolled infection, sepsis, or septic shock before being using the terlipressin to treat the HRS-1 patient.

In various embodiments, the terlipressin dosage is given as a continuous IV feed.

In one or more embodiments, the terlipressin dosage is 1 mg administered intravenously every 6 hours as a slow bolus injection over 2 minutes.

In various embodiments, the terlipressin dosage is not given as a bolus.

The terlipressin may be administered to the patient for up to 4 days, wherein the patient may be tested each day of the four days to determine whether the patient is responding to the terlipressin treatment. In various embodiments, a response to the terlipressin treatment may be indicated by a change in the patient's serum creatinine levels, where indication may be a reduction in SCr of at least 25% from baseline. In various embodiments, the terlipressin may be administered for at least 4 days.

At 260, the amount of serum creatinine change is determined after 4 days of treatment with terlipressin, and the treatment with terlipressin continued if the serum creatinine level has improved. In various embodiments, a sufficient improvement in serum creatinine levels after 4 days of treatment is indicated by a decrease of at least 1.0 mg/dl in serum creatinine level, or a decrease of about 1.7 mg/dL in serum creatinine level.

In various embodiments, the patient receives terlipressin for an additional 3 days to 8 days if improvement was exhibited over the previous 1 to 4 days. In various embodiments, the patient receives terlipressin for an additional 3 days to 4 days if improvement was exhibited over the previous 1 to 4 days.

In various embodiments, the administration of terlipressin to the patient is continued for an additional 3 days to 12 days beyond the initial 4 days if the patient exhibits a decrease in the serum creatinine level. In various embodiments, administration of terlipressin to the patient may be continued until at least one SCr value<1.5 mg/dL is obtained. In various embodiments, the duration of treatment may be extend to a maximum of 15 days or 16 days if HRS reversal was first achieved on days 13 or 14, respectively. In various embodiments, the duration of treatment of a patient with terlipressin may be 1 to 28 days. In various embodiments, a decrease in the serum creatinine level may be indicated by a reduction in SCr of at least 1% or of at least 5% or at least 10% or at least 15% or at least 20% or at least 25% from baseline.

In one or more embodiments, the patient may have been administered albumin prior to beginning the terlipressin treatment protocol, and/or prior to the determination that the patient has HRS-1, at least two of the three response criteria. In various embodiments, albumin may be administered to a patient 7 days to 2 days before starting administration of terlipressin to the patient. In various embodiments, the albumin treatment comprises administering 1 gram albumin per 1 kg of patient weight up to a maximum of 100 g per day of albumin to a patient. In various embodiments, albumin may be administered in the range of about 20 g/day to about 50 g/day, where the albumin may be administered for the time period that the patient is administered terlipressin.

A non-limiting embodiment of a method of treating HRS-1 patients exhibiting at least two of three response criteria with terlipressin comprises administering to a patient in need of such treatment a dosage of terlipressin in the range of 2.0 mg to 12.0 mg per day for 1 to 28 days, or in the range of 2.0 mg to 4.0 mg per day for 1 to 7 days, wherein the dosage may be administered as a continuous IV feed or as a slow bolus injection.

Embodiments of the present disclosure also relate to treating patients with HRS-1 and meeting two or more response criteria with one dose of terlipressin every six hours, where the dose is in the range of about 0.5 mg to 2.0 mg, for 3 to 8 days to achieve reversal of the HRS-1.

Embodiments of the present disclosure also relate to initiating terlipressin treatment within 48 hours of determining that a patient is presenting with HRS-1 and at least two of three response criteria, but without sepsis, septic shock, or uncontrolled infection.

Another aspect of the present disclosure relates to a method of distributing a pharmaceutical product.

In one or more embodiments, the method of distributing comprises supplying terlipressin to a medical provider, where the medical provider may be responsible for treating a patient suffering from type 1 hepatorenal syndrome. In various embodiments, the patient does not have overt sepsis, septic shock, or uncontrolled infection. In various embodiments, the method includes providing a recommendation to the medical provider to treat the patient suffering from type 1 hepatorenal syndrome that does not have overt sepsis, septic shock, or uncontrolled infection and having at least two of (1) a white blood cell count (WBC) is less than 4,000 cells/mm³ or greater than 12,000 cells/mm³, (2) a heart rate of greater than 90 beats per minute (BPM), or (3) an HCO₃<21 mmol/L, with terlipressin in an amount effective to reduce SCr. In one or more embodiments, the medical provider follows the recommendation and administers a treatment to the patient suffering from HRS-1, but not overt sepsis, septic shock, or uncontrolled infection and having at least two of (1) a white blood cell count (WBC) is less than 4,000 cells/mm³ or greater than 12,000 cells/mm³, (2) a heart rate of greater than 90 beats per minute (BPM), or (3) an HCO₃<21 mmol/L, with terlipressin in an amount effective to reduce SCr.

The efficacy of terlipressin versus placebo in achieving verified HRS-1 reversal may be more pronounced among the subgroup of patients with systemic inflammatory response syndrome (SIRS). Inflammatory cytokines have been implicated in the pathogenesis of HRS-1. Without being limited to any one theory, terlipressin, through its ability to reduce portal pressure, may decrease the extent of bacterial translocation across the gut wall of patients with decompensated cirrhosis, with consequent reduction in endotoxemia and decrease in the production of pro-inflammatory cytokines, hence making it easier for the patients to respond to the hemodynamic effects of terlipressin.

FIG. 3 shows the unexpected results produced by an exemplary treatment protocol.

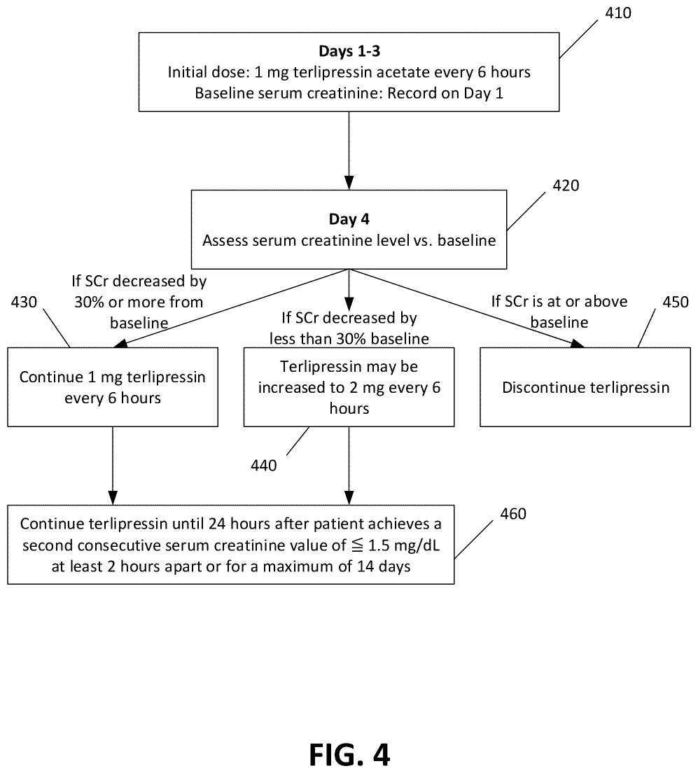

An aspect of the present disclosure relates to methods of treating and/or reversing HRS-1. As shown in FIG. 4, an exemplary embodiment of a method of treating an adult patient with HRS-1 via an embodiment of a terlipressin treatment protocol.

In various embodiments, a patient, who is initially identified as having end stage liver disease, for which treatment with a vasodilator may provide an improvement in renal function, may be tested to determine the extent of the patient's cirrhosis and renal failure. In an embodiment, the patient to be treated is an adult patient that has been diagnosed with HRS-1.

In one or more embodiments, the method of treating an adult patient with type 1 hepatorenal syndrome (HRS-1) includes assessing a baseline serum creatinine (SCr) level prior to administration of terlipressin to the patient, initiating dosing of about 0.5 mg to about 1 mg of terlipressin to the patient every 6 hours by IV for 1-3 days, assessing a serum creatinine level in the patient at day 4±1 day from initiating dosing; and administering a modified dosage of terlipressin based on a comparison of the assessed serum creatinine level at day 4±1 day and the baseline serum creatinine level. In some embodiments, the method may further include continuing administration until 24 hours after two consecutive serum creatinine levels of ≤1.5 mg/dl at least 2 hours apart for a maximum of 14 days.

In one or more embodiments, the terlipressin dosage may be in the range of about 0.1 mg to about 10 mg, about 0.5 mg to about 10 mg, or 0.5 mg to about 5.0 mg, or 0.5 mg to about 2.0 mg, or 0.5 mg to about 1.0 mg, about 0.85 mg to about 1.7 mg, or about 1.0 mg to about 2.0 mg per single administration.

In an embodiment, the terlipressin administered may be terlipressin acetate. The terlipressin acetate dosage may be administered to the patient in the range of about 0.5 mg to about 2.0 mg. In various examples, the terlipressin acetate dosage may be about 0.5 mg, about 1 mg, about 1.5 mg, about 2 mg, about 2.5 mg, about 3 mg, about 3.5 mg, or about 4 mg.

The terlipressin may be prepared for injection as a white to off-white lyophilized powder in a single-dose vial for reconstitution at a dosage of 0.85 mg terlipressin (equivalent to 1 mg terlipressin acetate). In some embodiments, the terlipressin acetate dosage may be given at an initial dose of about 0.5 mg or about 1 mg. In at least one example, dosing may be initiated with 1 mg terlipressin acetate. In other embodiments, the terlipressin dosage may be modified after a period of time administering the initial dose. In at least one example, the modified dosage may be about 2 mg terlipressin acetate.

In various embodiments, the injections may be administered intravenously as slow bolus injections over 2 minutes, where the dose may be repeated every four to six hours. In one or more embodiments, the injections may be administered to the patient over about four hours to about six hours as an IV drip.

In an example, an initial terlipressin dosage is administered to the patient in the range of about 0.5 mg to about 1.0 mg, every 4 to 6 hours, as a series of single doses, so that the patient receives a single dose in the range of about 0.5 mg to about 1.0 mg of terlipressin followed by another single dose 4 to 6 hours later. In various embodiments, a patient may receive 4 to 6 doses over a 24 hour period, where each dose is in the range of about 0.5 mg to about 1.0 mg. In various embodiments, the total dosage does not exceed 4.0 mg over a 24 hour period. In some examples, the terlipressin dosage may be about 0.85 mg or about 1.0 mg terlipressin acetate.

At step 410, in some embodiments, a baseline serum creatinine level may be measured before administration of terlipressin on day 1. Then, an initial dose of terlipressin may be administered to the patient with HRS-1. In an example, the initial dose of terlipressin may about 0.5 mg to about 1.0 mg, and it may be administered every 6 hours for about 1-3 days. In at least one example, the initial dosage may be about 1.0 mg terlipressin acetate (i.e. 0.85 mg terlipressin).

At step 420, on day 4±1 day of administration (e.g. after a minimum of 10 doses), the serum creatinine level may be assessed and compared to the baseline level. In various embodiments, the patient that is being administered the terlipressin is assessed at least once during the days 1 to 4±1 day of administration to determine if the patient is responding to the terlipressin. In various embodiments, the patient may be tested once at the end of 3 or 4 days of administration of the terlipressin. In some examples, the serum creatinine level may be continually assessed (e.g. daily) until administration is discontinued. In various embodiments, the dosage administered to the patient may be adjusted based upon the measured serum creatinine level(s). In various embodiments, a patient being administered terlipressin may have their serum creatinine levels monitored for the entire time period that the patient is receiving terlipressin. In one or more embodiments, the patient's serum creatinine level may be tested every day, or every other day, or every third day, or every fourth day, to confirm that the patient is still responding positively to the terlipressin treatment.

The serum creatinine levels may be measured by any of the methods known in the art, for example, the Jaffe reaction using alkaline picrate. The GFR may be measured directly by clearance studies of exogenous markers, such as inulin, iohexol, iothalamate, and Cr51-EDTA, or by estimated glomerular filtration rate (eGFR) using creatinine testing methods that are traceable to a reference method based on isotope dilution-mass spectrometry (IDMS).

In various embodiments, the patient's creatinine levels are assessed to determine if there has been a reduction in the patient's serum creatinine, where a reduction in serum creatinine levels of about 1.0 mg/dl or greater, or in the range of about 1.0 mg/dL to about 2.0 mg/dl, or a reduction of about 1.7 mg/dl from the patient's initial baseline value indicates an improvement in renal function and that the patient is responding to the terlipressin. In some examples, the assessed serum creatinine level may be 30% or more less than the baseline serum creatinine level, may be between 1% and 29% less than the baseline serum creatinine level, or may be 0% or greater than the baseline serum creatinine level. At steps 430, 440, and 450, a modified dosage of terlipressin may then be administered based on the comparison of the assessed serum creatinine level at day 4±1 day and the baseline serum creatinine level.

At step 430, if the assessed SCr level decreased by 30% or more from the baseline SCr level at day 4±1 day, the about 0.5 mg to about 1.0 mg dosage of terlipressin may be continued to be administered to the patient every 6 hours. For example, the modified dosage may be the same as the initial dosage (e.g. 0.5 mg to 1.0 mg) if the assessed SCr level decreased by 30% or more from the baseline SCr level.

At step 440, if the assessed SCr level has decreased, but by less than 30% from the baseline level at day 4±1 day, the dosage of terlipressin may be increased to about 1.0 mg to about 2.0 mg every 6 hours. For example, the modified dosage may be about 0.1 mg to about 2.0 mg of terlipressin every 6 hours (±30 min) (8 mg/day) if the assessed SCr level has decreased, but by less than 30% from the baseline level. In at least one example, the modified dosage may be The assessed dose may not be increased from the initial dose if the subject had coronary artery disease; or in the clinical setting of circulatory overload, pulmonary edema, or treatment-refractory bronchospasm. In various embodiments, if dosing was interrupted due to a non-ischemic adverse event, terlipressin may be restarted at the same or lower dose (i.e., 0.5 to 1 mg q6h).

At step 450, if the assessed SCr level is at or above the baseline SCr level at day 4±1 day, the administration of terlipressin may be discontinued. For example, the modified dosage may be a discontinuation of administering terlipressin if the assessed SCr level is at or above the baseline SCr level.

At step 460, administration of terlipressin may be continued until 24 hours after the patient achieves a second consecutive serum creatinine value of ≤1.5 mg/dl at least 2 hours apart or for a maximum of 14 days. In various embodiments, the dosage may be repeated every four to six hours for a time period of one or more days until the patient shows recovery, or until the patient no longer shows improvement. In various embodiments, the duration of treatment of a patient with terlipressin may be 1 to 14 days. In various embodiments, the terlipressin may be administered for at least 4 days. In various embodiments, the patient is administered terlipressin for up to 14 days unless the patient experiences an adverse event. In various embodiments, the terlipressin may be administered for at least 3 days, at least 4 days, at least 5 days, at least 6 days, at least 7 days, at least 8 days, at least 9 days, at least 10 days, at least 11 days, at least 12 days, at least 13 days, or at least 14 days. In some examples, the terlipressin may be administered to the patient for a time period in the range of about 2 days to about 14 days, or for a time period in the range of about 4 days to about 8 days. In various embodiments, the time period is in the range of about 7 days. In various embodiments, the terlipressin treatment may be continued until there is a complete response.

In one or more embodiments, a treatment protocol comprising dosages of terlipressin surprisingly provides reversal of one or more complicating factors, such as vasodilation, and reduces mortality from the associated complications within a 90 day window starting with treatment.

Treatment of the patient may include an improvement in renal function. An improvement in renal function is indicated by a reduction in SCr of at least 25% or 30% from baseline, reversal of HRS (defined as a decrease in SCr level to ≤1.5 mg/dl), and/or confirmed HRS reversal (defined as two serum creatinine values of ≤1.5 mg/dl at least 48 hours apart)).

In one or more embodiments, the patent is alive at day 90 after initiating terlipressin treatment. For example, a patient that experiences HRS reversal, verified HRS reversal, and/or greater than 30% improvement in SCr after receiving terlipressin may have at least a 60%, 65%, or 70% likelihood of being alive at day 90. In other embodiments, the patient is alive at day 90 post-liver transplant after initiating terlipressin treatment. For example, a patient that experiences HRS reversal, verified HRS reversal, and/or greater than 30% improvement in SCr after receiving terlipressin may have at least a 35%, 40%, or 45% likelihood of being alive at day 90.

In various embodiments, the adult patient with HRS-1 also is SIRS positive. In one or more embodiments, a patient with uncontrolled infection, sepsis, or septic shock is excluded from the terlipressin treatment.

In one or more embodiments, the patient is also up to a maximum of 100 g per day of albumin each day that the patient is treated with terlipressin. In some examples, the patient may continue to be administered albumin after terlipressin has been discontinued.

The percentage of patients who may achieve verified HRS reversal may be significantly higher with terlipressin than with placebo. In some examples, the patients administered terlipressin may achieve two consecutive SCr values of 1.5 mg/dL or less at least 2 hours apart while receiving treatment by day 14 or discharge. This demonstrates a robust and clinically significant improvement in renal function. In additional examples, the patients administered terlipressin may achieve an absence of renal replacement therapy (RRT) for at least 10 days, which emphasizes the durability of this improvement in renal function. The durability of HRS reversal with terlipressin may also persist to at least day 30 without the need for RRT. In other examples, the patient administered terlipressin may achieve survival for at least 10 days, which establishes the effect of treatment on a key clinical outcome of initial survival. Terlipressin may be superior to placebo in inducing a response across all levels of baseline SCr, with the response rate to terlipressin inversely related to the baseline SCr.

Renal replacement therapy poses particular challenges to patients with HRS-1 and advanced acute-on-chronic liver failure, and the lower rate of RRT and longer survival without RRT in the terlipressin group is clinically relevant. This significantly reduced need for RRT extending into the post-transplant period in the terlipressin group has important clinical implications, as post-transplant RRT is a significant predictor of post-transplant morbidity with worse graft survival mortality, and resource utilization.

In additional aspects of the disclosure, a method of increasing survival of a patient having HRS-1 and low MAP includes administering an effective dose of terlipressin to a patient in need thereof about every 6 hours by intravenous (IV) bolus injection over about 2 minutes, where the dose is sufficient to yield an increase in MAP and decrease in heart rate in the patient.

In other aspects of the disclosure, a method of increasing survival of a patient having HRS-1 includes administering an effective dose of terlipressin to a patient in need thereof about every 6 hours by intravenous (IV) bolus injection over about 2 minutes, where the dose is sufficient to yield an increase in the diastolic, systolic and MAP, and decrease in heart rate in the patient. In further aspects of the disclosure, a method of increasing survival of a patient having HRS-1 includes administering an effective dose of about 0.5 mg to about 2 mg terlipressin acetate to a patient in need thereof about every 4 to 10 hours by intravenous (IV) bolus injection over about 1 to 5 minutes, where the dose is sufficient to yield an increase in MAP and decrease in heart rate in the patient.

Terlipressin may cause adverse effects generally consistent with its mechanism of action (MOA) and class effects with the possibility of an increased risk of certain serious adverse event. Patient selection is extremely important when employing terlipressin. Efficacy and safety outcomes in patients with a serum creatinine ≥5 mg/dl and/or a model end stage liver disease (MELD) score ≥35 at the time of terlipressin initiation may be a tipping point for clinical outcomes. For example, SCr ≥5 mg/dl, MELD score ≥35, and/or acute-on-chronic liver failure (ACLF) Grade ≥3 may be a threshold of advanced disease presentation, decreased kidney function response, and increased adverse events. Clinicians may consider avoiding terlipressin in late-presenting HRS-1 or in advanced acute on chronic liver failure, where the likelihood of benefit is low. Use of terlipressin in patients with SCr ≥5 mg/dl may be considered only when the anticipated benefit to the patient outweighs the potential risk. In other embodiments, use of terlipressin in patients with at least about SCr ≥5 mg/dl may be considered only when the anticipated benefit to the patient outweighs the potential risk. The adverse events may include ischemic or respiratory symptoms that may lead to serious or fatal outcomes. For example, one of the possible respiratory symptoms is serious respiratory failure, which may be a major safety concern. In one embodiment, the risk of respiratory failure may not be reliably predicted and managed. There may be multiple potential causes of respiratory failure. Other adverse events may be ischemia, pneumonia, or sepsis without a clear mechanism. The risk of fluid overload and associated albumin use may complicate the clinical presentation and management of the event.

The management of adverse events, side effects, and undesirable symptoms for the current invention may include a variety of mitigation strategies. In one embodiment, the method of the present invention comprises the mitigation strategy of actively managing fluid overload during therapy. Actively managing fluid overload may comprise reducing or discontinuing the administration of albumin, other fluids, and/or judicious use of diuretics. For clarity, reducing the administration of albumin may comprise lowering the dose or interrupting the dose of albumin. If fluid overload persists, the method of treating may further comprise reducing or discontinuing terlipressin treatment. Management of fluid overload by reducing or discontinuing the administration of albumin, other fluids, and/or judicious use of diuretics may also occur wherein the patient additionally has respiratory failure, severe kidney disease, pulmonary edema, dyspnea, tachypnea, ischemia, or a combination thereof.

Actively managing fluid overload may comprise terlipressin dose alteration, for example, if symptoms persist. Actively managing fluid overload may also comprise terlipressin dose reduction, dose interruption, or dose discontinuation (i.e., treatment discontinuation), if symptoms, side effects, or adverse events persist. In another embodiment, the method of the present invention comprises the mitigation strategy of monitoring oxygen saturation during therapy. Monitoring oxygen saturation via pulse oximetry may identify patients at risk of developing serious respiratory adverse events.

Patients with acute-on-chronic liver failure (ACLF) Grade ≥3 and/or serum creatinine >5 mg/dl treated according to the currently claimed invention may be at significant risk for serious or fatal respiratory failure. In one embodiment, a mitigation strategy may be to stop or discontinue treatment in patients with serum creatinine ≥5 mg/dl or patients with ACLF Grade ≥3. Another mitigation strategy in the present invention is to stop or discontinue treatment in patients with a hepatic encephalopathy score ≥3. Another mitigation strategy to use in the present invention is to exclude from treatment (i.e., exclusion criteria) patients with serum creatinine ≥5 mg/dl or a hepatic encephalopathy score ≥3, and/or an ACLF Grade ≥3. Stated differently, the present invention also includes an embodiment where only patients are treated (i.e., inclusion criteria) if they have a serum creatinine <5 mg/dl, or a hepatic encephalopathy score <3, and/or an ACLF Grade <3. In some embodiments, terlipressin may be administered to a patient with less than a critical level or threshold level of serum creatinine. In some examples, the critical level or threshold level may be about 5 mg/dl. In at least one example, terlipressin may be administered to a patient if the patient has a serum creatinine level of <5 mg/dl.

In some embodiments, the duration of terlipressin treatment may be about 6 days to about 7 days in patients with presenting SCr <5 mg/dL as compared to about 6 days to about 11 days in patients with a presenting SCr ≥5 mg/dl and treated with terlipressin. There may be no significant difference between the groups in the proportion of patients who receive antibiotics or albumin.

In additional embodiments, a patient with a serum creatinine level of <5 mg/dl and administered a dose of terlipressin may have a decreased likelihood of adverse events, an increased overall survival, a decreased overall ICU, non-ICU, or hospital stay, increased likelihood of complete response, and/or an increased likelihood of a partial response as compared to a patient with a serum creatinine level of ≥5 mg/dl administered terlipressin. A complete response may be when the patient's SCr level has decreased to ≤1.5 mg/dl. In some examples, terlipressin may be continued to be administered to the patient until the patient's serum creatinine level is ≤1.5 mg/dl. In other examples, terlipressin may be continued to be administered to the patient until 24 hours after two consecutive measured SCr levels of ≤1.5 mg/dl at least 2 hours apart. A partial response may be when the patient's SCr level has decreased ≥20%, or preferably ≥30%, but is >1.5 mg/dL. In an example, terlipressin may be continued to be administered to the patient until the patient's serum creatinine level has improved by greater than 20%.

Higher presenting SCr may be linked to poorer efficacy in patients with HRS-1. For example, patients with SCr ≥5 mg/dl may be significantly more likely to develop adverse events compared to those with a presenting SCr <5 mg/dl when administered terlipressin. In some examples, a complete response may be achieved in about 50% to about 60% of patients with presenting SCr <5 mg/dl as compared to about 10% to about 20% in patients with presenting SCr ≥5 mg/dL when administered terlipressin. In other examples, a partial response may be achieved in about 50% to about 60% of patients with presenting SCr <5 mg/dl as compared to about 15% to about 25% in patients with presenting SCr ≥5 mg/dL when administered terlipressin. In additional examples, patients with presenting SCr ≥5 mg/dL may be more likely to develop fluid overload or pulmonary edema (about 20% to about 30%) and multi-organ failure (about 25% to about 35%) compared to patients with a SCr <5 mg/dl (about 10% to about 20% and about 5% to about 10%, respectively) when administered terlipressin. Overall survival may be significantly better in patients with presenting SCr <5 mg/dL than in patients with presenting SCr ≥5 mg/dl when administered terlipressin. In further examples, patients with SCr <5 may have a significantly shorter ICU stay of about 0.5 to about 1.5 days as compared to about 5 days to about 10 days for patients with presenting SCr ≥5 mg/dL when administered terlipressin. In yet further examples, patients with SCr <5 may have a significantly shorter non-ICU stay of about 20 days to about 25 days as compared to about 30 days to about 40 days for patients with presenting SCr ≥5 mg/dL when administered terlipressin. In even further examples, patients with SCr <5 may have a significantly shorter total length of hospital stay of about 20 days to about 25 days as compared to about 40 days to about 45 days for patients with presenting SCr ≥5 mg/dL when administered terlipressin.

A further mitigation strategy in the present invention is to stabilize patients with respiratory events. Among other factors, the present invention may further comprise managing fluid overload and pneumonia prior to treatment. The mitigation strategies that may be used as part of the present invention may result in reduced adverse events, a reduced risk of mortality, a reduced incidence of mortality, and combinations thereof. Reduced risk of mortality or reduced incidence of mortality may include overall survival (e.g., measured as alive at Day 90 after beginning treatment).

Higher baseline MELD scores may be linked to poorer efficacy (e.g. poor survival) in patients with HRS-1. For example, patients with a baseline MELD score of greater than 35 may be significantly more likely to develop adverse events compared to those with a baseline MELD score of less than 35 when administered terlipressin. Patients may be excluded from treatment if the patient is listed for liver transplant with a MELD score ≥35.

In some embodiments, provided herein is a method of treating a patient with HRS-1 by administering a dose of terlipressin to the patient by IV injection when the patient has a baseline model end stage liver disease (MELD) score of less than 35. In some examples, the patient is listed for transplant at baseline and has a baseline MELD <35. In other examples, the patient is not listed for transplant at baseline and has MELD score <35 or >35. The administration of terlipressin to this subset of patients may lead to increasing overall survival of the patient, decreasing an overall ICU or hospital stay of the patient, increasing a complete response of the patient, and/or increasing a partial response of the patient. In some embodiments, the overall survival at day 90 of a patient with a baseline MELD score <35 treated with terlipressin may be increased by about 5% to 50%, about 5% to 15%, about 10% to 20%, about 25% to 35%, about 30% to 40%, about 35% to 45%, or about 40% to 50% as compared to placebo and/or as compared to a patient with a baseline MELD score ≥35 treated with terlipressin. In some embodiments, the transplant-free survival at day 90 of the patient with a baseline MELD score <35 treated with terlipressin may be increased by about 5% to 50%, about 5% to 15%, about 10% to 20%, about 25% to 35%, about 30% to 40%, about 35% to 45%, or about 40% to 50% as compared to placebo and/or as compared to a patient with a baseline MELD score ≥35 treated with terlipressin.

In some embodiments, the patient with a MELD score <35 may also have severe kidney disease, pulmonary edema, dyspnea, or a combination thereof.

The method may further include acquiring the baseline MELD score of the patient, acquiring a serum creatinine (SCr) level in the patient prior to administering the dose of terlipressin to determine a baseline SCr level, and/or determining the patient's acute-on-chronic liver failure (ACLF) grade. In some embodiments, the terlipressin may not be administered if the baseline SCr is >5 mg/dl and/or ACLF Grade is >3. In other embodiments, the method may include discontinuing administration or reducing the dose of terlipressin in patients with SCr ≥5 mg/dl and/or an ACLF Grade ≥3. In additional embodiments, a dose of terlipressin may be administered to the patient if the patient's baseline MELD score is <35 and the baseline SCr level is <5 mg/dl. Patients with a baseline MELD score of less than 35 may further be monitored for a SCr level of greater than 5 mg/dl and or an ACLF grade of greater than or equal to 3 for discontinuation of the terlipressin administration. In some examples, the patient is listed for transplant at baseline and has a baseline MELD <35. In other examples, the patient is not listed for transplant at baseline and has MELD score <35 or ≥35.

In some embodiments, administering terlipressin to patients with a baseline MELD score of <35 may decrease the patient's risk of mortality (i.e. increase survival). The patient may be listed for transplant and have a MELD score of <35. In an embodiment, treatment of patients on the transplant list at baseline with a baseline MELD score of <35 may further aid in not compromising a patient's place on a transplant list. In some embodiments, the patient may have a reduced risk of their place on a transplant list being compromised or impacted due to the administration of terlipressin.

Only patients who are actively listed for transplantation can receive an available organ. Their wait time from being listed to receiving a liver transplant is dependent on other factors (i.e., MELD score, geographic region, blood type) and may vary from days to months based on their MELD score. Excluding patients who are likely to receive a transplant during or shortly after terlipressin treatment may reduce the risk that such patients will not receive a transplant due to any adverse effects of terlipressin therapy, e.g., respiratory failure. This also prioritizes patients who are at highest risk of dying while waiting for a liver and therefore directs livers to those most in need.

For example, a patient on the transplant list with a MELD score of ≥35 may be high on the transplant list and therefore may have a faster treatment time by waiting for the liver transplant rather than be treated with terlipressin. Therefore, not treating patients listed for a liver transplant with a baseline MELD score ≥35 may allow the patient to maintain their spot on the transplant list and receive a transplant quicker than if they were first treated with terlipressin. In at least one embodiment, the patient may only be treated if the patient belongs to a patient population that has median waiting time from listing to transplant of about 5.6 months or greater. In another embodiment, the patient may be excluded from treatment if the patient belongs to a patient population that has median waiting time from listing to transplant of 0.23 months (approximately 7 days) or less. In additional embodiments, the patient's ICU stay, non-ICU stay, and/or total length of hospital stay may be shortened due to the patient having a baseline MELD score <35, a baseline SCr level of <5 mg/dl, and/or an SCLF grade of <3.

In some embodiments, the terlipressin administration is continued until there is a complete response or a partial response. In some embodiments, discontinuing administration or reducing the dose of terlipressin occurs in patients with respiratory failure. For example, administering terlipressin to patients with a baseline MELD score of <35 may reduce the likelihood of the patient having respiratory failure as compared to patients with a baseline MELD score of ≥35.

The dose of terlipressin administered may be 0.5 mg to about 2 mg terlipressin acetate and may be administered every 4 to 10 hours by IV bolus injection over about 1 to 5 minutes. In at least one example, the terlipressin may be administered every 6 hours by IV bolus injection over 2 minutes.

In some embodiments, the method may further include monitoring the patient's oxygen saturation during treatment with the terlipressin. The monitoring of the oxygen saturation may decrease the occurrence of adverse events. For example, a method of treating an HRS patient may include obtaining a baseline oxygenation level (SpO₂) via pulse oximetry, administering a dose of terlipressin to the patient by IV injection if the patient is not experiencing hypoxia, and monitoring the patient's SpO₂ during treatment with the terlipressin. The patient's SpO₂ may be obtained at baseline prior to the first dose of terlipressin and then may be monitored at least 3 times a day, at least 4 times a day, at least 5 times a day, at least 6 times a day, or continuously during administration of terlipressin. The patient's oxygen saturation (SpO₂) may be monitored for hypoxia. An SpO₂ value of <90% may be indicative of some degree of hypoxia. Fraction of inspired oxygen (FiO₂) may also be monitored as an indication of pulmonary function. In an example, FiO₂≥0.36 may be an indication of compromised pulmonary function. In an embodiment, the method may further include discontinuing administration or reducing the dose of terlipressin if hypoxia is detected.

In additional embodiments, the method may further include monitoring the patient for fluid overload during treatment with the terlipressin. The terlipressin dose may then be reduced or discontinued if fluid overload develops. Diuretics may also be administered to the patient if fluid overload develops.

In an embodiment, the method may further include measuring the SCr level in the patient during administration of terlipressin. The administration may then be continued until the patient's SCr level is ≤1.5 mg/dl or until the patient experiences greater than 20% improvement in SCr. In some examples, administration is continued until the patient experiences greater than 30% improvement in serum creatinine.

Also provided herein is a method of treating a patient with HRS-1 by narrowing the population of eligible patients for treatment to a mitigated population to reduce the risks selected from the group consisting of respiratory failure, serious adverse events, death, and combinations thereof and then administering a dose of terlipressin to the patient of the mitigated population by IV injection. The mitigated population excludes patients with baseline ACLF Grade 3, baseline serum creatinine ≥5 mg/dL, and/or patients listed for transplant at baseline with a baseline MELD ≥35. It was surprising that this mitigated population had improved survival, lower incidence of respiratory failure, and lower pre-transplant mortality as compared to an overall population treated with terlipressin. The treatment of HRS-1 patients having a baseline ACLF Grade 0-2, baseline SCr <5 mg/dL, and/or be listed for transplant at baseline with a baseline MELD <35 may favorably impact the incidence of liver transplantation and minimize the risk that a patient listed for liver transplant will be precluded from receiving a transplant due to a potential adverse effect of terlipressin.

Treating this subset of HRS patients (e.g. mitigated population) leads to a higher rate of verified HRS reversal, lower incidence of renal replacement therapy (RRT), and favorable RRT-free survival with terlipressin compared with placebo. For example, this subset of treated patients may have a reduction in risks compared with the overall population, including lower incidence of respiratory failure, overall mortality, and pre-transplant mortality. In some embodiments, the overall survival, verified HRS reversal, HRS reversal, durability of HRS reversal, HRS reversal in SIRS subgroup, and/or verified HRS reversal without HRS recurrence by day 30 of a patient in the mitigated population treated with terlipressin may be increased by about 5% to 50%, including about 5% to 15%, about 10% to 20%, about 25% to 35%, or about 30% to 40% as compared to the overall population treated with terlipressin. In other embodiments, the overall survival, verified HRS reversal, HRS reversal, durability of HRS reversal, HRS reversal in SIRS subgroup, and/or verified HRS reversal without HRS recurrence by day 30 of a patient in the mitigated population treated with terlipressin may be increased by about 5% to 100%, including about 5% to 15%, about 10% to 20%, about 25% to 35%, about 30% to 40%, about 35% to 45%, about 40% to 50%, about 50% to 60%, about 60% to 70%, about 70% to 80%, about 80% to 90%, or 90% to 100% as compared to the overall population or the mitigated population given placebo. In additional embodiments, there may be an about 5% to 50%, about 5% to 15%, about 10% to 20%, about 25% to 35%, about 30% to 40%, about 35% to 45%, or about 40% to 50% decrease in the incidence of RRT for patients alive by day 90 for patients in the mitigated population treated with terlipressin as compared to the overall population or mitigated population given placebo. In various embodiments, patients in the mitigated population treated with terlipressin may have an increased likelihood of survival without RRT by day 14, day 30, or day 60 as compared to patients in the overall population treated with terlipressin. For example, patients in the mitigated population treated with terlipressin may have an about 2% to about 20%, about 5% to 15%, or about 10% to 20% increase in the likelihood of survival without RRT by day 14, day 30, or day 60 as compared to patients in the overall population treated with terlipressin. In further embodiments, patients in the mitigated population treated with terlipressin may have an increased likelihood of survival without RRT by day 14, day 30, day 60, or day 90 as compared to patients in the overall population treated with placebo or the mitigated population treated with placebo. For example, patients in the mitigated population treated with terlipressin may have an about 2% to about 40%, about 5% to 10%, about 10% to 20%, about 20% to 30%, or about 30% to 40% increase in the likelihood of survival without RRT by day 14, day 30, day 60, or day 90 as compared to patients in the overall population treated with placebo or the mitigated population treated with placebo.

### EXAMPLES

### Example 1

A randomized, placebo-controlled, double-blind study (“REVERSE”) was conducted to evaluate the efficacy of terlipressin in HRS type 1. The objective of the study was to determine the efficacy and safety of intravenous terlipressin compared with placebo in the treatment of adult patients with HRS type1 receiving intravenous albumin. Men and women aged 18 years or older having cirrhosis, ascites, and a diagnosis of HRS type 1 based on the 2007 International Club of Ascites (ICA) diagnostic criteria (Salerno F, Gerbes A, Gines P, Wong F, Arroyo V., Diagnosis, prevention and treatment of hepatorenal syndrome in cirrhosis, Gut. 2007; 56:1310-1318) were eligible for participation. Patients with an SCr level>2.5 mg/dl and either a doubling of SCr within 2 weeks or a change in SCr levels over time indicating a trajectory with a slope equal to or greater than that of a doubling within 2 weeks were enrolled.

Exclusion criteria were intended to product a patient sample limited to individuals with functional renal impairment secondary to cirrhosis and ascites, who could safely be administered terlipressin and who could be expected to survive through the active study period. Among the original exclusion criteria was an exclusion criterion for patients with systemic inflammatory response syndrome (SIRS), defined as the presence of 2 or more of the following findings: (1) temperature>38° C. or <36° C.; (2) heart rate>90/min; (3) respiratory rate of >20/min or a PaCO₂ of <32 mm Hg; (4) white blood cell count of >12,000 cells/μL or <4,000/g L. This was based on the concern of enrolling patients with uncontrolled infection. However, it was also recognized that patients with decompensated liver disease frequently have SIRS criteria in the absence of uncontrolled infection or sepsis, and that the presence of 2 or more SIRS criteria is associated with a poor prognosis (Thabut, et al., “Model for End-Stage Liver Disease Score and Systemic Inflammatory Response Are Major Prognostic Factors in Patients with Cirrhosis and Acute Functional Renal Failure,” HEPATOLOGY, Vol. 46, No. 6, December 2007, pp. 1872-1882). Furthermore, the IAC criteria for the definition of HRS type 1 allows for patients with ongoing bacterial infection, but not sepsis or uncontrolled infection, to be considered as having HRS type 1 (as opposed to renal dysfunction associated with infection) (Salerno F, Gerbes A, Gines P, Wong F, Arroyo V., Diagnosis, prevention and treatment of hepatorenal syndrome in cirrhosis, Gut. 2007; 56:1310-1318). The trial protocol required 2 days of anti-infective therapy for documented or suspected infection, allowing enrollment where any SIRS criteria were felt to be most likely explained by underlying hepatic decompensation or other non-infection clinical circumstances. Patients with overt sepsis, septic shock, or uncontrolled infection were excluded. This approach was felt to minimize the chances of enrolling patients at high risk for serious infection while not unduly restricting the enrollment of subjects with HRS type 1.

The patients selected for treatment clinically met the criteria for HRS type 1, where ICA criteria for HRS type 1 allows for patients with ongoing bacterial infection, but not sepsis, to be considered as having HRS type 1, as opposed to renal dysfunction associated with infection. A diagnosis of HRS was not made where the patient remained with obvious manifestations of uncontrolled infection despite antibiotic treatment.

During the active study period treatment with the blinded study drug continued until at least two SCr values<1.5 mg/dl were obtained at least 48 hours apart, or up to 14 days. Duration of treatment was allowed to extend to a maximum of 15 or 16 days if HRS reversal was first achieved on days 13 or 14, respectively. Patients in the active treatment group received terlipressin 1 mg intravenously every 6 hours as a slow bolus injection over 2 minutes. Criteria for dose increases, study discontinuation, treatment resumption and treatment completion during the active study period were provided for. The dosing regimen for patients in the placebo (6 mL lyophilized mannitol solution) group was identical to the terlipressin regimen. The follow-up period began after the end of study treatment and concluded 90 days after the start of study treatment. Survival, renal replacement therapy, and transplantation were assessed.

The SIRS subgroup of patients in this study was defined as any subject with ≥2 of 3 criteria available from the study database which included: (1) WBC<4 or >12 cells/μL; (2) HR>90 bpm and (3) HCO₃<21 mmol/L. The latter criterion represented an approximation of the SIRS criterion PaCO₂ of <32 mm Hg. This approximation was derived from the observed HCO₃ in subjects with HRS in whom a PaCO₂ value was available and the calculated HCO₃ in subjects with decompensated liver disease and PaCO₂ of <32 mm Hg. The non-SIRS subgroup was defined as subjects with <2 criteria described above. Terlipressin response was analyzed in the SIRS and non-SIRS subgroups to determine whether SIRS status had any effect on terlipressin efficacy.

A total of 196 patients were enrolled in the study. Of the 196 patients enrolled, 58 were initially identified as having ≥2 SIRS criteria, including WBC<4 or >12 cells/μL, HR>90 bpm, and HCO₃<21 mmol/L, wherein this population was identified as the SIRS subgroup. Based on the criteria defining the SIRS subgroup, baseline WBC and heart rate were slightly higher, and bicarbonate slightly lower, in the SIRS subgroup compared to the non-SIRS and overall study populations. The results of the analysis are shown in FIG. 3.

It was also recognized that patients with decompensated liver disease frequently have SIRS criteria in the absence of uncontrolled infection or sepsis, and that the presence of two or more SIRS criteria is associated with a poor prognosis.

In one or more embodiments, reversal of HRS is indicated by a decrease in SCr level to ≤1.5 mg/dl, and confirmed reversal of HRS is defined as two SCr values of ≤1.5 mg/dl at least 48 hours apart.

As shown in FIG. 3, patients identified as having HRS-1 and at least two of the three criteria for SIRS on a terlipressin treatment protocol exhibited a statistically significant increase in confirmed reversal of HRS (32.1% vs. 3.3%, p<0.005), HRS reversal (42.9% vs. 6.7%, p<0.002) and renal function (change from baseline in SCr, mg/dl, −1.7 vs. −0.5, p<0.0001) compared to placebo. In contrast, in the group of patients having HRS-1 and fewer than two of the SIRS criteria, confirmed reversal of HRS vs. placebo was 14.5% vs. 17.4%, HRS reversal vs. placebo was 15.9% vs. 18.8%, and renal function change vs. placebo was −0.8 vs. −0.7 mg/dL. These results indicate that the presence of two or more of the SIRS criteria indicates that the patient is more likely to have a positive response to treatment with terlipressin.

In addition, in the treatment groups, patients with HRS-1 and two or more SIRS criteria showed an overall survival rate comparable to patients that were suffering from HRS-1, but did not have at least two of the three criteria for SIRS (57.1% vs. 58%).

### Example 2

A randomized, placebo-controlled, double-blind study (“CONFIRM”) was conducted to evaluate the efficacy of terlipressin in HRS type 1. The objective of the study was to characterize the efficacy and safety of terlipressin plus albumin versus albumin alone for the treatment of HRS-1 in patients with well-defined HRS-1. The study used the similar inclusion and exclusion criteria as described in Example 1.

In particular, HRS-1 was defined based on modified prior criteria outlined by the International Club of Ascites (ICA), as rapidly deteriorating renal function to SCr ≥2.25 mg/dL, with actual or projected doubling of SCr within 2 weeks, without improvement in renal function (<20% decrease in SCr 48 hours after both diuretic withdrawal and albumin-fluid challenge) in adult patients with cirrhosis and ascites. Subjects were randomized 2:1 to terlipressin (1 mg IV every 6 hours) or placebo, plus albumin in both groups. Treatment was continued to Day 14 unless the following occurred: verified HRS reversal (VHRSR), renal replacement therapy (RRT), liver transplantation (LT) or SCr at or above baseline (BL) at Day 4. VHRSR, the primary endpoint, was defined as 2 consecutive SCr values≤1.5 mg/dL, at least 2 hours apart, with subjects alive without RRT for at least 10 days after the second SCr $1.5 mg/dL; HRS reversal (HRSR) was a decrease in SCr to ≤1.5 mg/dL. Secondary end points included HRS reversal (any SCr value 1.5 mg/dL or less during treatment), HRS reversal without RRT by day 30, HRS reversal in patients with systemic inflammatory response syndrome, and verified HRS reversal without recurrence by day 30.

The patients were at least 18 years of age, with cirrhosis, ascites, and rapidly progressive renal failure, with a SCr doubling to at least 2.25 mg/dl within 14 days. Major exclusion criteria included SCr of greater than 7.0 mg/dl, one or more large-volume paracenteses of 4 L or more within 2 days of randomization, evidence of parenchymal renal diseases or obstructive uropathy, or presence of sepsis and/or uncontrolled bacterial infection. Patients with severe cardiovascular disease or recent (within 4 weeks) renal replacement therapy (RRT) were excluded.

300 subjects were enrolled in the study. Of the 300 subjects, 199 were randomized to terlipressin and 101 to placebo (albumin alone). Patients were stratified by qualifying SCr (less than 3.4 mg/dl or 3.4 mg/dL or greater) and pre-enrollment large-volume paracentesis (at least one single event of 4 L or greater, or less than 4 L within 3 to 14 days prior to randomization). Concomitant albumin was administered in 82.9% of patients in the terlipressin group (165 of 199; mean [SD] total dose of 199.4 [146.8] g) versus 91.1% (92 of 101; mean [SD] dose of 239.5 [183.6] g) in the placebo group (P=0.06). One hundred forty-five patients (72.9%) in the terlipressin group and 72 (71.3%) in the placebo group had received prior midodrine and octreotide.

Demographic and BL clinical characteristics were similar between treatment groups. For example, the two treatment groups had similar average age, weight, height, sex distribution, ethnicity distribution, race distribution, presence of alcoholic hepatitis, baseline serum creatinine, large volume paracentesis (LVP) randomization strata, baseline model end stage liver disease (MELD) score, baseline Child-Pugh score, baseline white blood cell count, baseline bilirubin, baseline mean arterial pressure (MAP), baseline heart rate, baseline blood urea nitrogen (BUN), baseline bicarbonate (HCO₃) or carbon Dioxide (CO₂), baseline temperature, baseline respiratory rate, baseline acute on chronic liver failure (ACLF) grade, baseline chronic liver failure-sepsis organ failure assessment (CLIF-SOFA) score and presence of prior conditions/treatments such as esophageal variceal hemorrhage (EVH) banding, pneumonia, urinary tract infection (UTI), spontaneous bacterial peritonitis (SBP), and receipt of albumin. The proportion of patients in each group who underwent LT was 23.1% for terlipressin and 28.7% for placebo.

A baseline SCr value was assessed before the patients received the assigned treatment. Patients received blinded assigned treatment (terlipressin or placebo) 1 mg administered intravenously over 2 minutes every 6 hours (±30 minutes). In keeping with current guidelines, it was strongly recommended that albumin (1 g/kg to a maximum of 100 g, on day 1 and 20 to 40 g/day thereafter) be administered to all subjects. If SCr decreased <30% from the baseline value on Day 4, after a minimum of 10 doses of study drug, dose increase to 2 mg every 6 hours (±30 minutes) (8 mg/day) was mandated, except in subjects with coronary artery disease or in the setting of circulatory overload, pulmonary edema, or bronchospasm. Dose resumption was permitted after interruption for adverse events except for cardiac or mesenteric ischemia, for which treatment was permanently discontinued.

The primary efficacy end point was the incidence of verified HRS reversal, defined as the percentage of patients with two consecutive SCr values no greater than 1.5 mg/dL at least 2 hours apart, while remaining alive without RRT for at least 10 days after achieving verified HRS reversal, while excluding SCr values after RRT, transjugular intrahepatic portosystemic shunt, liver transplant, or open-label vasopressor from primary end point analysis. 58 patients (29.1%) treated with terlipressin achieved verified HRS reversal versus 16 (15.8%) treated with placebo (P=0.01).

Secondary efficacy end points included incidence of HRS reversal, defined as the percentage of patients with an on-treatment SCr value of 1.5 mg/dl or less; durability of HRS reversal, defined as the percentage of patients with HRS reversal without RRT to day 30; incidence of HRS reversal among patients with systemic inflammatory response syndrome; and incidence of verified HRS reversal without HRS recurrence by day 30. 36.2% of patients treated with terlipressin achieved HRS reversal versus 16.8%, (P<0.001) treated with placebo. 31.7% of patients treated with terlipressin achieved HRS reversal without RRT by day 30 versus 15.8% (P=0.003) treated with placebo. The reduction in RRT requirement with terlipressin appears to extend into the post-liver transplant period, with only 9 of 46 patients (19.6%) requiring RRT post-transplant, significantly less than what was observed in the placebo group (13 of 29 patients or 44.8%), (P=0.04). A slightly lower percentage of patients in the terlipressin group received a liver transplant (23.1% [46 of 199]) compared with placebo (28.7% [29 of 101]). 24.1% of patients treated with terlipressin achieved verified HRS reversal without recurrence by day 30 versus 15.8%, (P=0.09) treated with placebo.

132/300 (44%) of subjects met systemic inflammatory response syndrome (SIRS) criteria, as defined in Example 1. Patients with overt sepsis, septic shock, or uncontrolled infection were excluded. In the SIRS subgroup, 84 patients were treated with terlipressin per the protocol in Example 1 and 48 patients were give albumin only (placebo).

Some baseline values of SIRS patients treated with terlipressin or placebo are shown in Table 1 below.

TABLE 1

| | SIRS Subgroup | SIRS Subgroup |
|---|---|---|
| | Terlipressin n = 84 | Placebo n = 48 |
| BL SCr | 3.5 (0.98) | 3.7 (1.06) |
| mg/dL(mean(SD) | (2.3-6.2) | (2.2-6.1) |
| (range) | | |
| MELD mean (SD) | 33.8 (6.27) | 33.5 (6.74) |
| CPT score mean (SD) | 10.2 (1.82) | 10.3 (2.26) |

As seen in Table 2, 33.3% of patients with SIRS and treated with terlipressin experienced HRS reversal, as compared to only 6.3% of the SIRS patients given placebo. In addition, 26.2% of patients with SIRS and treated with terlipressin experienced verified HRS reversal, as compared to only 4.2% of the SIRS patients given placebo.

TABLE 2

| | SIRS Subgroup | | |
|---|---|---|---|
| | (N = 132) | | |
| | Terlipressin | Placebo | |
| | (N = 84) | (N = 48) | |
| | n (%) | n (%) | P value |
| HRS Reversal (n, %) | 28 (33.3) | 3 (6.3) | <.001 |
| 95% CI | (0.2, 0.4) | (0.0, 0.1) | — |
| Verified HRS Reversal | 22 (26.2) | 2 (4.2) | <.001 |

Table 3 shows transplant-free survival up to 90 days for subjects with HRS reversal and/or greater than 30% improvement in serum creatinine (SCr) compared to subjects with no HRS reversal and no more than 30% improvement in SCr in the SIRS subgroup of the intent-to-treat population. 45.5% of the SIRS subgroup treated with terlipressin having HRS reversal and/or at least 30% improvement in SCr were alive and transplant-free at day 90, as compared to 28.6% for placebo. 72.7% of the SIRS subgroup treated with terlipressin having HRS reversal and/or at least 30% improvement in SCr were alive at day 90, as compared to 57.1% for placebo.

TABLE 3

| | SIRS Subgroup: | | | |
|---|---|---|---|---|
| | HRS Reversal and/or Greater | | | |
| | than 30% | | | |
| | Improvement in SCr | | | |
| | Terlipressin | | Placebo | |
| | N | Parameter | N | Parameter |
| Transplant-free Survival up to | | | | |
| 90 Days | | | | |
| Survival Estimate | 33 | 0.680 | 7 | 0.536 |
| Alive and Transplant-free at | 33 | 15 (45.5) | 7 | 2 (28.6) |
| Day 90 (n, %) | | | | |
| Overall Survival up to 90 Days | | | | |
| Survival Estimate | 33 | 0.727 | 7 | 0.571 |
| Alive at Day 90 (n, %) | 33 | 24 (72.7) | 7 | 4 (57.1) |

A significantly higher proportion of subjects in the SIRS subgroup of the intent-to-treat (ITT) population in the terlipressin group (33.3%) than in the placebo group (6.3%) achieved HRS reversal. Among subjects with SIRS at baseline, the incidence of RRT was lower in the terlipressin group than in the placebo group at all follow-up timepoints (Table 4). The mean cumulative frequency of RRT through Day 90 were 7.3 days in the terlipressin group compared to 15.2 days in the placebo group.

TABLE 4
RRT in Subjects with SIRS at Baseline

| | Terlipressin | Placebo |
|---|---|---|
| | (N = 84) n (%) | (N = 48) n (%) |
| Subjects with RRT by Day 14 | 20 (23.8) | 20 (41.7) |
| Subjects with RRT by Day 30 | 23 (27.4) | 21 (43.8) |
| Subjects with RRT by Day 60 | 25 (29.8) | 22 (45.8) |
| Subjects with RRT by day 90 | 27 (32.1) | 22 (45.8) |
| Cumulative Frequency of RRT | | |
| through Day 30 | | |
| N | 23 | 22 |
| Mean (SD) | 4.5 (3.63) | 7.7 (6.56) |
| Cumulative Frequency of RRT | | |
| through Day 90 | | |
| N | 27 | 22 |
| Mean (SD) | 7.3 (9.08) | 15.2 (15.27) |

The median time to first RRT was 6.0 days in the terlipressin group and 5.5 days in the placebo group. The transplant-free survival estimate up to Day 90 was higher in the terlipressin group than the placebo group for subjects with SIRS at baseline (Table 5).

TABLE 5
Transplant-free Survival up to 90 Days with Events for Death and Transplant in
Subjects with SIRS at Baseline by Treatment Group
(Intent-to-treat Population, SIRS Subgroup)

| | Terlipressin | | Placebo | | |
|---|---|---|---|---|---|
| | N | Parameter | N | Parameter | P-value |
| Transplant-Free | | | | | |
| Survival up to 90 Days | | | | | |
| Survival Estimate | 84 | 0.290 | 48 | 0.192 | 0.363 |
| Median Days of | 84 | 15.0 | 48 | 18.0 | |
| Survival | | | | | |
| Alive and Transplant- | 84 | 26 (31.0) | 48 | 10 (20.8) | |
| Free at Day 90 (n, %) | | | | | |

After Day 30, the overall survival estimate up to Day 90 was slightly higher in the terlipressin group than the placebo group for subjects with SIRS at baseline. For the SIRS subgroup, overall survival of subjects out to 90 days was analyzed to compare differences between subjects who achieved verified HRS reversal or >30% improvement in serum creatinine and those who did not). For the SIRS subgroup, in both treatment groups up to Day 90, the survival estimate was higher in responders than nonresponders. The RRT-free survival estimate up to Day 90 was higher in the terlipressin group than the placebo group for subjects with SIRS at baseline.

A lower percentage of subjects with SIRS at baseline in the terlipressin group was admitted to the ICU: 14 (16.7%) subjects in the terlipressin group compared with 12 (25.0%) subjects in the placebo group. SIRS subjects in the terlipressin group had a shorter mean length of stay in the ICU (6.3 days) than in the placebo group (12.1 days).

Applying strict criteria defining HRS-1, the study demonstrated a significant reversal of worsening renal function in cirrhotic patients treated with terlipressin plus albumin when compared to those treated with albumin alone, including patients with SIRS criteria. This response was durable and associated with less need for early RRT. Therefore, terlipressin is effective in improving renal function and achieving HRS reversal in patients with HRS-1 and progressive advanced liver disease.

### Example 3

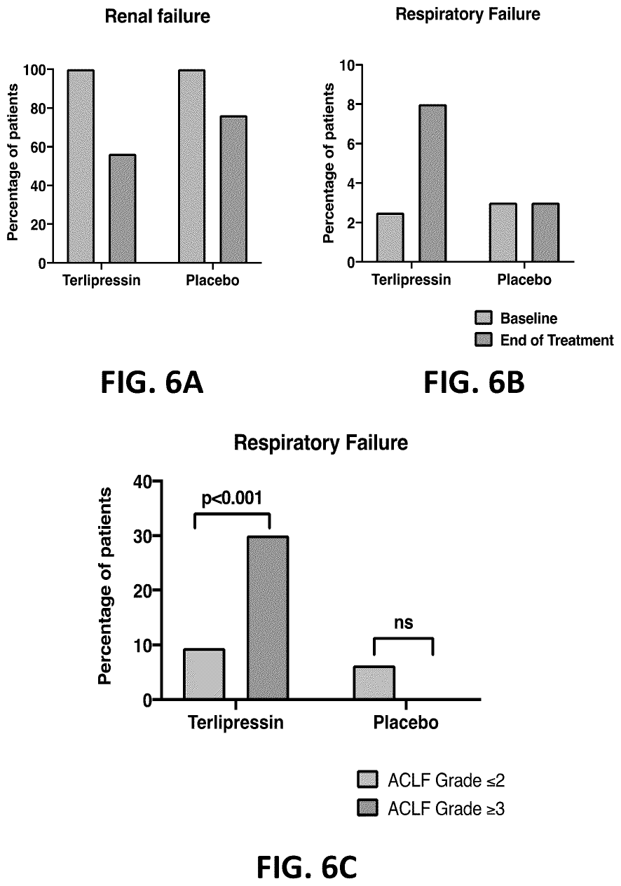

As seen in FIGS. 6A-6B, there was an increased incidence of respiratory failure in those patients who received terlipressin, but not in those who received placebo. In FIG. 6B, the incidence of respiratory failure was based on CLIF-SOFA score definition of respiratory failure. Therefore, this study assessed in patients with HRS1 whether the presence of severe or grade 3 ACLF according to the EASL-CLIF system at baseline was a risk factor for the development of respiratory failure with terlipressin use.

The study was a double-blind placebo-controlled trial. 300 patients with HRS-1 were randomized 2:1 to receive terlipressin versus placebo in 1-2 mg i.v. bolus injections 6 hourly, both with albumin. HRS-1 was defined as rapidly rising serum creatinine (SCr) to ≥2.25 mg/dl in <14 days without response to volume challenge or evidence of structural renal disease.

All patients were assessed for organ failure (OF) per EASL-CLIF criteria and then classified into grades of acute-on-chronic liver failure (ACLF). All patients had at minimum grade 1 ACLF due to the presence of HRS-1; grades 2 and 3 ACLF represent 2 or 3 OFs respectively. Patients were separated into grade ≤2 and grade 3 ACLF subgroups. The effects of terlipressin vs. placebo on the incidence of RF between these subgroups were compared. Table 6 shows the patient demographics and Table 7 shows the baseline clinical and laboratory parameters in the patients of the study.

TABLE 6
Patient Demographics

| | ACLF Grade ≤2 | | | |
|---|---|---|---|---|
| | (n = 242) | | ACLF Grade 3 (n = 58) | |
| | Terlipressin | Placebo | Terlipressin | Placebo |
| n | 159 | 83 | 40 | 18 |
| Age (yrs) | 55.7 ± 11.0 | 54.6 ± 11.7 | 47.6 ± 10.8 | 47.8 ± 10.8 |
| M:F (n) | 94:66 | 48:33 | 26:14 | 10:8 |
| SIRS no. (%) | 63 (39.4%) | 35 | 22 (55.0%) | 12 (66.7%) |
| | | (33.2%) | | |
| Alcoholic | 56 (35.6%) | 27 | 25 (62.5%) | 12 (66.7%) |
| hepatitis | | (33.3%) | | |
| no. (%) | | | | |
| Infection in prior | 67 (41.9%) | 37 | 21 (52.5%) | 11 (61.1%) |
| 14 days: no. (%) | | (45.7%) | | |
| Child-Pugh score | 9.6 ± 1.8 | 10.0 ± 1.9 | 11.6 ± 1.3 | 11.6 ± 1.5 |
| MELD score | 30.8 ± 6.3 | 31.6 ± 5.8 | 39.2 ± 1.8 | 38.3 ± 4.7 |

TABLE 7
Baseline Clinical & Laboratory Parameters

| | ACLF Grade ≤2 (n = 242) | | ACLF Grade 3 (n = 58) | |
|---|---|---|---|---|
| n | 159 | 83 | 40 | 18 |
| Heart rate | 77.8 ± 15.8 | 82.0 ± 15.1 | 84.9 ± 14.3 | 90.9 ± 12.1 |
| (beats/min) | | | | |
| MAP (mmHg) | 77.6 ± 11.8 | 77.4 ± 9.4 | 82.9 ± 12.2 | 79.1 ± 9.0 |
| WBC (x10⁹/l) | 8.9 ± 6.1 | 8.7 ± 5.2 | 11.3 ± 5.0 | 11.4 ± 6.2 |
| INR | 2.0 ± 0.67 | 2.0 ± 0.56 | 3.1 ± 0.86 | 4.3 ± 5.22 |
| Creatinine (mg/dl) | 3.5 ± 1.04 | 3.5 ± 1.04 | 3.5 ± 0.93 | 3.4 ± 0.96 |
| Bilirubin (mg/dl) | 9.4 ± 11.3 | 11.7 ± 12.5 | 27.1 ± 12.2 | 29.2 ± 19.8 |
| Prior Albumin (g) | 331 ± 171 | 369 ± 299 | 349 ± 164 | 391 ± 152 |

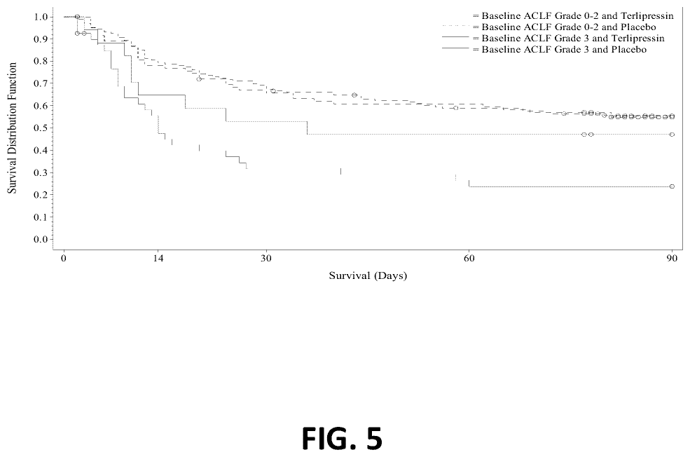

FIG. 5 is a graph showing 90-day survival of patients treated with and without terlipressin according to ACLF grade. Patients with an ACLF grade of 3 and treated with terlipressin had a lower likelihood of survival than patients with an ACLF grade of 0-2 and treated with terlipressin or patients treated with placebo regardless of ACLF grade. FIG. 6C shows the percent of patients with respiratory failure among those treated with terlipressin was similar to placebo for patients with an ACLF grade of 0-2. The percent of patients with respiratory failure in patients treated with terlipressin with an ACLF score of 3 was much higher than placebo. The rate of respiratory failure in FIG. 6C was based on reporting of respiratory failure during the study by the investigator.

Table 8 provides the odds ratio for additional baseline parameters as predictors of respiratory failure with terlipressin use.

TABLE 8
Predictors of Respiratory Failure with Terlipressin Use

| Parameter | Odds Ratio | P value |
|---|---|---|
| Baseline INR | 1.81 | 0.011 |
| Baseline MAP | 1.037 | 0.037 |
| Baseline SpO2 | 0.835 | 0.014 |

The results of Tables 4-6 indicate that terlipressin should be used with extreme caution in patients with HRS-1 and ACLF grade 3, especially in those with compromised oxygen saturation. Patients with low baseline SpO₂ are at risk for respiratory failure (RF) and increased mortality.

### Example 4

The following analyses were performed to evaluate the impact of the proposed mitigation strategy on the incidence of liver transplantation and pre-transplant mortality of subjects listed at baseline in two prior terlipressin studies (REVERSE [Example 1] and CONFIRM [Example 2]). The mitigated population excludes data from subjects with baseline acute-on-chronic liver failure Grade 3 and baseline serum creatinine >5 mg/dl, and subjects listed for transplant at baseline with baseline MELD ≥35.

The incidence of liver transplantation by Day 90 in the mitigated population in CONFIRM was slightly higher in the terlipressin group compared to the placebo group (24.2% vs. 22.5%) in contrast to the intent-to-treat (ITT) population where the incidence of liver transplantation in the terlipressin group was lower compared to placebo (23.1% vs. 28.7%) (Table 9).

TABLE 9
Incidence of Transplant by Day 90 in CONFIRM

| | Overall Population | | Mitigated Population | |
|---|---|---|---|---|
| | Terlipressin | Placebo | Terlipressin | Placebo |
| | n/N | n/N | n/N | n/N |
| | (%) | (%) | (%) | (%) |
| Received | 46/199 (23.1) | 29/101 | 32/132 (24.2) | 16/71 |
| Transplant | | (28.7) | | (22.5) |
| by Day 90 | | | | |

Similarly, the incidence of subjects listed at baseline who did not receive a transplant and died by Day 90 was substantially reduced from 21.4% (12 subjects) in the overall population to 11.4% (4 subjects) in the mitigated population. Of the 12 subjects listed at baseline in the overall population who did not receive a transplant and died by Day 90, 6 subjects were mitigated by excluding data in subjects with ACLF-3 and SCr ≥5 mg/dl and an additional 2 subjects were mitigated with the MELD ≥35 cut-off criteria (Table 10). Similar effects of the mitigation strategy in the terlipressin group were observed in the REVERSE study.

TABLE 10
Incidence of Patients Listed at Baseline Who did not Receive a Transplant
and Died by Day 90 by Population in CONFIRM and REVERSE

| | REVERSE + CONFIRM | | REVERSE | | CONFIRM | |
|---|---|---|---|---|---|---|
| | Terlipressin | Placebo | Terlipressin | Placebo | Terlipressin | Placebo |
| | n/N (%) | n/N (%) | n/N (%) | n/N (%) | n/N (%) | n/N (%) |
| Overall | 20/92 (21.7) | 8/59 (13.6) | 8/36 (22.2) | 8/39 (20.5) | 12/56 (21.4) | 0/20 (0.0) |
| Population | | | | | | |
| Population | 10/72 (13.9) | 7/44 (15.9) | 4/28 (14.3) | 7/30 (23.3) | 6/44 (13.6) | 0/14 (0.0) |
| ACLF ≤2 & | | | | | | |
| SCr <5 | | | | | | |
| Mitigated | 6/53 (11.3) | 6/35 (17.1) | 2/18 (11.1) | 6/25 (24.0) | 4/35 (11.4) | 0/10 (0.0) |
| Population | | | | | | |

Due to the limited number of donor organs available in the US, only about half of all wait-listed subjects in 2018 received a liver within one year and many patients die or are removed from the wait list without undergoing transplant (Kwong 2021). For example, 11.3% of all patients listed for liver transplant in 2016 died prior to receiving a transplant (Kwong 2021), and the 90-day pre-transplant mortality was 10.5% overall and 25.7% in patients with a MELD score of ≥35 (Nagai 2018).

The pre-transplant mortality in the mitigated population in the CONFIRM study and in the pooled dataset of the CONFIRM and REVERSE studies was consistent with the published data for all US transplant-listed patients. In aggregate, these analyses provide that in patients receiving terlipressin, the mitigation strategy favorably impacts the incidence of liver transplantation and minimizes the risk that a patient listed for liver transplant will be precluded from receiving a transplant due to a potential adverse effect of terlipressin.

In the mitigated population in the CONFIRM study, there was a clinically meaningful reduction in overall SAEs, respiratory failure SAEs, overall fatal AEs, fatal respiratory failure AEs, and overall mortality in the terlipressin group compared with the respective overall population (Table 11).

TABLE 11
Overview of Safety for the Overall and Mitigated Population in CONFIRM

| | Overall Population | | Mitigated Population | |
|---|---|---|---|---|
| | (% of Subjects) | | (% of Subjects) | |
| | Terlipressin | | Terlipressin | |
| | (N = 200) | Placebo (N = 99) | (N = 132) | Placebo (N = 71) |
| Serious Adverse | 65.0 | 60.6 | 61.4 | 59.2 |
| Events | | | | |
| Respiratory | 13.5 | 5.1 | 9.8 | 7.0 |
| failure | | | | |
| Any AE leading | 41.5 | 40.4 | 34.1 | 40.8 |
| to death up to 30 | | | | |
| days post- | | | | |
| treatment | | | | |
| Respiratory | 8.5 | 1.0 | 5.3 | 1.4 |
| failure | | | | |
| Death by Day 30 | 39.0 | 36.4 | 31.8 | 36.6 |
| Death by Day 60 | 47.0 | 41.4 | 40.2 | |
| Death by Day 90 | 51.0 | 44.4 | 44.7 | 45.1 |

There was a clinically meaningful reduction in the incidence of overall SAEs and SAEs due to respiratory failure in the mitigated population in CONFIRM (Table 11). The between-group difference in the incidence of respiratory failure SAEs in the overall study population of 8.4% (13.5%-5.1%) was reduced to 2.8% (9.8%-7.0%) in the mitigated population. For the terlipressin group, the incidence of overall SAEs in the mitigated population is lower than in the overall study population without mitigation (61.4% vs. 65.0%, respectively). In the mitigated population, the incidence of overall SAEs in the terlipressin group was similar to the placebo group (61.4% vs. 59.2%, respectively).

The incidence of overall AEs leading to death, fatal respiratory failure AEs, and overall mortality was substantially reduced in the mitigated population in CONFIRM. The between-group difference in the incidence of respiratory failure fatal AEs in the overall population of 7.5% (8.5%-1.0%) was reduced to 3.9% (5.3%-1.4%) in the mitigated population. The incidence of all AEs leading to death up to 30 days post-treatment for the terlipressin group in the mitigated population is lower than in the overall population without mitigation (31.8% vs. 39.0%, respectively) and also lower than in the placebo group in the mitigated population (31.8% vs. 36.6%, respectively).

The incidence of liver transplantation by Day 90 in the mitigated population was slightly higher in the terlipressin group compared with the placebo group (24.2% vs. 22.5%, respectively) in contrast to the overall population, where the transplantation rate was lower (23.1% vs. 28.7%, respectively; Table 8). Importantly, the pre-transplant mortality by Day 90 of subjects listed for transplant at baseline in the terlipressin group was reduced from 21.4% in the overall population to 11.4% in the mitigated population (Table 10) and is within the range of what is reported in all subjects listed for liver transplantation in the US.

The benefit of terlipressin therapy on verified HRS reversal demonstrated in the overall CONFIRM study population was also observed in the mitigated population (Table 12). The benefit of terlipressin over placebo was likewise preserved for the secondary endpoints. The impact of the primary endpoint on the key clinical outcomes of renal replacement therapy (RRT) and RRT-free survival was also maintained. Similar to overall population, in the mitigated population, the incidence of RRT was lower at all time points through Day 90 in the terlipressin group than in the placebo group.

TABLE 12
Overview of Efficacy for the Overall and Mitigated Population in CONFIRM

| | Overall Population (% of Subjects) | | | Mitigated Population (% of Subjects) | | |
|---|---|---|---|---|---|---|
| | Terlipressin | Placebo | | Terlipressin | Placebo | |
| | (N = 199) | (N = 101) | p-value | (N = 132) | (N = 71) | p-value |
| Primary endpoint | | | | | | |
| Verified HRS | 29.1 | 15.8 | 0.012 | 36.4 | 18.3 | 0.007 |
| Reversal | | | | | | |
| Secondary | | | | | | |
| endpoint | | | | | | |
| HRS Reversal | 36.2 | 16.8 | <0.001 | 44.7 | 18.3 | <0.001 |
| Durability of HRS | 31.7 | 15.8 | 0.003 | 38.6 | 18.3 | 0.003 |
| reversal | | | | | | |
| HRS reversal in | 33.3 | 6.3 | <0.001 | 41.2 | 3.3 | 0.001 |
| SIRS subgroup | | | | | | |
| Verified HRS | 24.1 | 15.8 | 0.092 | 28.8 | 18.3 | 0.103 |
| reversal without | | | | | | |
| HRS recurrence | | | | | | |
| by Day 30 | | | | | | |
| Key Clinical | | | | | | |
| Outcome | | | | | | |
| Subjects with RRT | 30.3 | 46.4 | — | 30.1 | 41.0 | — |
| for alive subjects | | | | | | |
| by Day 90 | | | | | | |

The mitigated population is at a similar risk for undergoing RRT as the overall population as evidenced by the proportion of subjects who received RRT up to Day 90 (Table 13). In these subjects, RRT-free survival remained numerically favorable in the terlipressin group compared with the placebo group.

TABLE 13
Subjects Alive Without RRT Though Day 90 in the Overall Population and the
Mitigated Population in CONFIRM

| | Overall Population | | Mitigated Population | |
|---|---|---|---|---|
| | (% of Subjects) | | (% of Subjects) | |
| | Terlipressin | Placebo | Terlipressin | Placebo |
| | (N = 199) | (N = 101) | (N = 132) | (N = 71) |
| Subjects Alive without | 114/146 (78.1) | 47/77 (61.0) | 87/107 (81.3) | 36/55 (65.5) |
| RRT by Day 14 | | | | |
| Subjects Alive without | 89/121 (73.6) | 37/65 (56.9) | 70/90 (77.8) | 29/45 (64.4) |
| RRT by Day 30 | | | | |
| Subjects Alive without | 75/105 (71.4) | 32/60 (53.3) | 57/79 (72.2) | 25/41 (61.0) |
| RRT by Day 60 | | | | |
| Subjects Alive without | 69/99 (69.7) | 30/56 (53.6) | 51/73 (69.9) | 23/39 (59.0) |
| RRT by Day 90 | | | | |

In the CONFIRM overall population, the overall survival estimate up to Day 90 was numerically lower in the terlipressin group than in the placebo group (48.2% vs. 53.5%, respectively). In contrast, in the mitigated population, the overall survival estimate up to Day 90 was similar in the terlipressin group compared with the placebo group (55.3% vs 54.9%, respectively).

The risk mitigation strategy preserved the beneficial effects of terlipressin as demonstrated in the overall CONFIRM study population (i.e., statistical significance for primary endpoint, lower incidence of RRT, and favorable RRT-free survival with terlipressin compared with placebo). In the mitigated population, there was also a clinically meaningful reduction in risks compared with the overall population, including lower incidence of respiratory failure, overall mortality, and pre-transplant mortality. Therefore, the mitigated population represents a population where the benefits of terlipressin treatment outweigh the risks in this rare complication of liver disease associated with a high medical need and no current FDA-approved or proven pharmacological treatments.

Although the disclosure herein has been described with reference to particular embodiments, it is to be understood that these embodiments are merely illustrative of the principles and applications of the present disclosure. It will be apparent to those skilled in the art that various modifications and variations can be made to the devices, systems, and methods of the present disclosure without departing from the spirit and scope of the disclosure. Thus, it is intended that the present disclosure include modifications and variations that are within the scope of the appended claims and their equivalents.

Reference throughout this specification to “one embodiment,” “certain embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

What is claimed is:

1. A method of treating an adult patient aged 18 years or older with hepatorenal syndrome comprising:
obtaining a baseline oxygenation level (SpO₂) from the patient via pulse oximetry;
administering a dose of a composition comprising terlipressin to the patient by intravenous (IV) injection if the patient's SpO₂ at baseline is greater than 90%;
monitoring the patient's SpO₂ during administration of the composition at least 3 times a day; and
discontinuing administration or reducing the dose of the composition if the patient's SpO₂ during treatment is less than 90%,
wherein the patient is only treated if the patient belongs to a patient population that has median waiting time from listing to transplant of 5.6 months or greater.
